SEQUENCE LISTING (1 sequence)

SEQ ID NO: 1 - protein, 9 aa, Haemophilus influenzae
ASNENMETM

(19) United States
(12) Patent Application Publication
(10) Pub. No.: US 2006/0177468 A1
(43) Pub. Date: Aug. 10, 2006

(54) Delivery vehicles, bioactive substances and viral vaccines

(75) Inventors: Peter D. Katsikis, Merion Station, PA (US); Elizabeth S. Papazoglou, Yardley, PA (US); Margaret A. Wheatley, Media, PA (US); Nadarajan Sundar Babu, Philadelphia, PA (US); Alina Boestcanu, Willow Grove, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Philadelphia Health and Education Corporation (d/b/a Drexel University College of Medicine; (DUCOM)

(21) Appl. No.: 11/327,674

(22) Filed: Jan. 5, 2006

Related U.S. Application Data
(60) Provisional application No. 60/641,502, filed on Jan. 5, 2005.

(51) Int. Cl. A61K 39/145

(52) U.S. Cl. 424/209.1

(57) ABSTRACT

The invention relates to compositions and methods for the safe delivery of a bioactive agent to an animal. Preferably, the bioactive agent is a vaccine, and more preferably, the bioactive agent is a virus.

### BACKGROUND OF THE INVENTION

The present invention relates to novel virus vaccines exemplified by, but not limited to an influenza virus vaccine. The invention further relates to novel compositions and methods for safe delivery of bioactive substances to animals, preferably vertebrates, that if delivered to a vertebrate in an aerosol or free form, may have an adverse effect on the vertebrate.

Influenza A virus causes the common flu and is the leading viral cause of mortality in the United States (Yewdell et al., 2002, Curr. Opin. Microbiol. 5:414). This is largely due to the fact that immunocompromised individuals are susceptible to a more severe and deadly case of the flu compared to healthy people. Influenza virus is an RNA virus that belongs to the Orthomyxoviridae family. The viral genome comprises eight single-stranded RNA segments which encode the following proteins: Two surface glycoproteins named hemagglutinin (HA) and neuraminidase (NA), the M2 ion-channel protein, the M1 matrix protein, the nucleoprotein associated with viral RNA, and three RNA polymerases (PA, PB1 and PB2) (Horimoto et al., 2005, Nat. Rev. Microbiol. 3:591-600). Due to the error-prone nature of the RNA genome, the influenza virus accumulates mutations in the two major surface proteins: HA and NA. Based on the antigenic differences that exist among HA or NA within the type A influenza virus group sixteen different HA and nine different NA subtypes have been identified. A second level of genetic diversity exists by virtue of the capacity of the RNA fragments to undergo reassortment within an infected host, thus creating viruses containing RNA segments of human and animal origin (Zhou et al., 1999, J. Virol. 73:8851-8856).

As a result of the antigenic variability of influenza virus, the composition of the viral envelope proteins, hemagglutinin (H) and neuraminidase (N), is always changing. This variability results in emerging strains of virus that often have increased efficiency for transmission either within an animal species, or between species. More importantly, the generation of new strains of virus leads to pandemics because there is little or no immunity directed against the new strains within the susceptible human population (Enserink, 2004, Science 306:392). For this reason, the 1918-1919 “Spanish flu” was the most deadly pandemic ever, killing an estimated 100 million people worldwide.

Recently, concern has been rising about the transmission of influenza virus between species. The influenza virus strains that were found to circulate in the human population predominantly express HA subtype 1, 2 or 3 and NA subtype 1 or 2. The influenza virus pandemic in 1918 (Spanish influenza) was caused by an H1N1 virus. This devastating pandemic infection was followed by others throughout the century, for example, Asian flu (H2N2) in 1957 and Hong Kong flu (H3N2) in 1968. Genetic variants of H1N1 and H3N2 viruses continue to infect humans and cause epidemics every year. The other subtypes of HA and NA of influenza virus type A are maintained in aquatic birds. Therefore, through the process of reassortment, a new HA subtype could be introduced in the human population from the avian reservoir and consequently cause a pandemic infection. The inefficient replication of avian influenza viruses in humans was considered a major obstacle to the emergence of pandemic infections.

Previously it was thought that human and avian influenza viruses reassorted only in pigs because this was the only species that could be infected by both types of viruses. However, in 1997, the first case of direct avian to human transmission was identified. This virus infected eighteen people and killed six of them (Enserink, 2004, Science 306:392; Kaiser, 2004, Science 306:394). Since 1997, there have been six more outbreaks of three strains of avian flu in the human population, and the H5N1 strain has emerged as being the most common and deadly strain (Kaiser, 2004, Science 306:394). This cross species transmission poses the risk that a pandemic influenza strain is emerging against which humans are not protected by any of the currently available vaccines.

In view of the increasing concern over the possibility of a new influenza virus pandemic, there have been enhanced efforts to discover an influenza virus vaccine that is capable of protecting humans against more than one strain of virus. These efforts are also directed to the discovery of therapeutic molecules that have the effect of treating influenza virus infection by preventing replication of the virus (Cox et al. 2004, Scand. J. Immunol. 59:1; Kaiser, 2004, Science 306:395). This research has been aimed at developing DNA based vaccines, RNA interference molecules that would act to protect humans against influenza virus strains, and the development of new antiviral drugs active against influenza virus (Kaiser, 2004 Science 306:395; Epstein et al., 2002, Emerg. Infect. Dis. 8:796; Tompkins et al., P 2004, PNAS, 101:8682; Tumpey et al., 2004, PNAS 101:3166).

Currently there are two influenza vaccines that are commercially available for humans use in the U.S. One is a killed virus vaccine that is administered as an intramuscular injection, and the other is an attenuated vaccine that is administered as a nasal spray. Both of these vaccines elicit anti-influenza virus antibodies that neutralize subsequent infection by the same virus. However, emerging strains of virus which express variant antigenic epitopes may not be recognized by existing antibodies. Thus, a different vaccine must be prepared and administered each year.

Antiviral drugs are also available that act by interfering with different steps of the virus life cycle. The virus enters the host cell by receptor-mediated endocytosis. Inside the endosome, low pH triggers the fusion of viral and endosomal membranes and the M2 ion channel allows an influx of H+ ions which leads to release of viral genes into the cytoplasm. Two antiviral drugs, amantadine and rimantidine, block the M2 ion-channel thus impeding virus uncoating and RNA release into the cytoplasm. A second class of antiviral drugs acts on NA and interferes with virus packaging and budding of newly infectious particles from the cell. NA acts by removing sialic acid containing receptors on the cell surface such that newly generated virus particles do not aggregate with other virus particles or remain attached to the surface of the infected cell. Therefore, NA inhibitors, such as oseltamivir and zanamivir, cause the virions to remain attached to the membrane of the infected cell and prevent their attachment and therefore infection of other cells. These drugs may provide a method to contain virus spread in case of a pandemic infection despite the existence of naturally resistant strains to M2 and NA blockers (Monto and Arden, 1992, Clin. Infect. Dis. 15:362-367; Kiso et al., 2004, Lancet 364:759-765).

Keeping in mind that the next pandemic will likely be caused by a virus with a HA subtype that has not previously infected humans, new techniques for vaccine development are being tested. For example, reverse genetics is being used to clone the HA and NA genes of candidate strains into plasmids. Cells are then transfected with these plasmids along with the other six genes of a master donor strain. Thus, the virions produced by reverse genetics combine the expression of the relevant antigenic molecules HA and NA with characteristics of the donor strain (high yield, cold adapted, attenuated strain). Reverse genetics considerably reduces the amount of time required to obtain candidate vaccines (Webby et al., 2004, Lancet 363:1099-1103) However, in the event of a pandemic, the causative virus may reach the U.S. in less than one month (Enserink, 2004, Science 306:392-394). Thus, the time that is needed to develop an effective vaccine using this strategy may not be sufficient. Other drawbacks associated with reverse genetics is the difficulty associated with transfecting eight or more plasmids into one cell at the same time and the limited number of cell lines approved for vaccine manufacturing.

Another antiviral approach is the use of short interfering RNAs (siRNA) directed against conserved regions of the influenza virus genes. siRNAs are RNA duplexes that are 21-26 nucleotides in length and that can induce sequence-specific degradation of homologous mRNA. Intravenous delivery of siRNA specific for nucleoprotein or acidic polymerase was shown to protect mice from lethal challenge with influenza virus strains known to infect mice or with highly pathogenic avian strains such as H5 and H7 subtypes (Tompkins et al., 2004, PNAS 101:8682-8686). Although siRNA interferes with the influenza virus life cycle, major difficulties arise when this technology is adapted to humans. It is important that the siRNA sequence is not complementary to any human gene sequence and the siRNA must be expressed in sufficient levels in all infected cells in order to block the activity of viral genes. Overall, the new antiviral technologies may have long term promise for the development of new influenza virus vaccines or therapies. However, the immediate demand for the generation of millions of doses of vaccine in a very short period of time is not resolved using these technologies.

Beginning in 1997, there have been three more outbreaks of the highly pathogenic avian virus H5N1 in the human population: Hong Kong 2003, Vietnam 2004 and Thailand 2004 (Kaiser, 2004, Science 306:394-397). A vaccine directed against this influenza virus subtype is being produced and tested for efficacy and safety. It has been reported that the vaccine can protect humans against the H5N1 strain; however, the dose necessary for inducing protection (90 μg of purified killed virus or antigen) is double that used in the case of the common seasonal influenza virus vaccine. In addition, the H5N1 vaccine has to be administered twice to a human at four week intervals (Enserink, 2004, Science 309:996). In order to increase the efficacy of the anti-H5N1 vaccine and to be able to administer lower doses of antigen, different strategies have been proposed such as use of an adjuvant or changing the route of delivery of the virus (Schwartz and Gellin, 2005, J. Infect. Dis. 191:1207-1209).

As noted, currently available vaccines directed against influenza virus strains include an inactivated (killed) vaccine which is administered as an intramuscular injection, and an attenuated live vaccine that is administered as a nasal spray. Both of these vaccines result in the production of antiviral, or virus neutralizing antibodies (Cox et al., 2004, Scand. J. Immunol. 59:1). Emerging strains of the virus which express variant antigenic epitopes are not recognized by existing antibodies. Therefore, when this is the vaccine strategy of choice, a new vaccine must be developed and administered to humans each year (Cox et al., 2004, Scand. J. Immunol. 59:1; Kaiser, 2004, Science 306:395). If a pandemic strain of influenza virus is identified, it is anticipated that at least six months are required to develop an inactivated vaccine that would be effective against the new strain (Kaiser, 2004, Science 306:394; Kaiser, 2004, Science 306:395). In order to develop a vaccine that protects humans against a pandemic strain of influenza virus, the vaccine must not only be capable of eliciting an antibody response against the virus, but also optimally, the vaccine should induce a CD8+ T cell response that exhibits a broad spectrum specificity against several pandemic strains. While such a vaccine may not prevent actual infection by any one pandemic strain of virus, it should ameliorate the severity of the disease thereby reducing the morbidity and mortality following infection.

A critical issue for the development and successful use of any bioactive substance or vaccine is whether or not it is safe for use in animals and humans. The assessment of safety must be made at two levels. On the one hand, the bioactive substance or vaccine must not be toxic to the animal into which it is administered. On the other hand, personnel who handle the bioactive substance or vaccine, for example, those that administer the substance or vaccine, the substance or vaccine recipients, and others that might be present during administration, must not be at risk for any adverse effects caused by the bioactive substance or virus contained therein. The latter situation is particularly important if the bioactive substance is a toxin or the vaccine comprises a live virus. As will be apparent from the disclosure provided herein, this problem can be solved by encapsulating the bioactive substance or virus in a medium that prevents spread of virus by aerosolization. In view of this, the prior art disclosures of differing compositions are now reviewed herein.

Biocompatible gels have been studied and used extensively for drug delivery, cytokine delivery (Liu et al., 2003, Cancer Chemother. Pharmacol. 51:53-57), gene therapy (Schek et al., 2004, Molecular Therapy 9:130-138), and tissue engineering (Tsang and Bhatia, 2004, Adv. Drug Deliv. Res. 56:1635-1647). A number of biocompatible polymers have been used in vivo. The majority of recent investigations into vaccine encapsulation have emphasized the advantages of using pulsed or sustained release formulations in order to address the call by the World Health Organization for a single-step immunization (Aguado, 1993, Vaccine 11:596-597). Polymeric microcapsules that are capable of releasing antigen have been shown to induce an enhanced immune response in mammals for periods of time greater than six months (Pries and Langer, 1979, J. Immunol. Methods 28:193-197). Immunopotentiation by these formulations is believed to occur by either depot effect similar to aluminum salt adjuvants, or by delivery of the antigen directly to antigen presenting cells.

Hydrogels are generally defined as colloidal gels in which water is the dispersion medium. They are composed of polymers which are cross linked by a variety of different bonds that are either chemical or physical, such a ionic or hydrophobic interactions, or by hydrogen bonds. Alginate is a naturally occurring linear polysaccharide extracted from brown seaweed. It is composed of 1-4 linked α-L-guluronic and β-D-mannuronic acid residues. Different sources of alginate have different guluronic acid content, and this in turn affects the property of the alginate. Alginate can form hydrogels by reaction with divalent cations such as Ca²⁺, Ba²⁺ Sr²⁺ and the like, but not with Mg²⁺. Trivalent cations such as Al³⁺ and Fe³⁺ have also been used to form hydrogels from alginate. The general method of preparation of these hydrogels involves dropping a sodium alginate solution into a solution that contains the necessary crosslinking cations. Liposomes encapsulated in alginate have been studied for protein delivery (Wheatley et al., 1991, J. Applied Polymer Science 43:2123-2135; Dhoot and Wheately 2003, J. Pharmaceut. Sciences 92:679-689; U.S. Pat. No. 4,921,757) and several different cell lines including pancreatic islet cells (Lim and Sun, 1980, Science 210:908-910) and genetically engineered fibroblasts (Tobias et al., 2001, J. Neurotrauma 18:287-301; Cheng et al., 1998, Human Gene Therapy 9:1995-2003) have been encapsulated in alginate for therapeutic applications. In recent years, alginate has been investigated for use as a scaffold in tissue engineering (Kuo and Ma, 2001, Biomaterials 22:511-521). Alginate hydrogels with covalently coupled peptides have been studied as synthetic extracellular materials (Suzuki et al., 2000, J. Biomed. Materials Res. 50:405-409; Rowley et al., 1999, Biomaterials 20:45-53) and as a tissue bulking agent (Loebsack et al., 2001, J. Biomed. Materials Res. 57:575-581). It has been reported that ionically crosslinked alginates lose mechanical properties over time in vitro, presumably due to an outward flux of crosslinking ions into the surrounding medium (Shiochet et al., 1996, Biotechnology and Bioengineering 50:374-381). Methods for the ionotropic gelation of alginate include for example, those described in the following references: Wheatley et al. (1991, J. Appl. Pol. Sci. 44:2123; Dhoot and Wheatley (2003, J. Pharm. Sci. 92:679); and Dhoot et al. (2004, J. Biomed. Mater. Res. 71A:191).

A similar hydrogel can be formed from hyaluronic acid, also known as hyaluron, a polymer normally found in the body. Hyaluronic acid is a negatively charged linear polymer of D-glucuronic acid and N-acetyl-D-glucosamine formed when these compounds are exposed to multivalent cations (Balazs and Laurent, 1998, In: The Chemistry, Biology and Medical Applications of Hyaluronan and Its Derivatives, 325-336; Chen and Abatangelo, 1999, Wound Repair Regen. 7:79-89). Hyaluronic acid is known to be highly biocompatible, as evidenced by its frequent application in joint repair (Lim et al., 2000, J. Controlled Release 66:281-292; Prestwich et al., 1998, J. Controlled Release 53:93-103). There have been few studies on this compound for use in drug delivery because it rapidly dissolves at physiological pH (Campoccia et al., 1998, Biomaterials 19:2101-2127).

Delivery of drugs using collagen matrices gained its importance primarily due to its inherent biodegradability, weak antigenicity (Maeda et al., 1999, J. Controlled Release 62:313-324) and superior biocompatibility when compared with natural biopolymers such as albumin. Collagen matrices have been used as carriers for gene therapy (Cohen-Sacks et al., 2004, J. Controlled Release 95:309-320), controlled release of proteins (Fijioka et al., 1995, J. Controlled Release 33:307-315), antibodies (Fleming and Saltzman, 2001, J. Controlled Release 70:29-36), antibiotics (Verbukh et al., 1993, Collagen Shields Impregnated With Gentamicin-Dexamethasone As A Potential Drug Delivery Device, Elsevier Science Publishers) and for delivery of growth factors such as transforming growth factor-beta 2 (TGF-β2) (Schroeder-Tefft et al., 1997, J. Controlled Release 49:291-298) to mammals. A collagen matrix has been used to deliver an adenoviral vector encoding platelet-derived growth factor-B (AdPDGF-B) to a mammal (Chandler et al., 2000, Molecular Therapy 2:153-160), and was found to increase the expression of the encoded transgene in both in-vivo and in-vitro wound healing. Gu et al. (2004, Molecular Therapy 9:699-711) showed that an adenovirus encoding human platelet-derived growth factor-B delivered in a matrix of collagen induced an antibody response directed against the adenovirus. A T cell response was not noted. Adenovirus-containing collagen gels have also been disclosed by others for delivery of genes to an animal (Schek et al., 2004, Mol. Ther. 9:130). However, these gels were not used in a vaccine setting for the specific purpose of inducing a protective immune response in an animal. Instead, the absence of an immune response to the virus was desired in these studies because the presence of a response was expected to result in rejection of the virus by the animal and therefore the desired effect of gene delivery would be nullified. To effect controlled release of a drug, collagen fibrils must be crosslinked to form a matrix. Individual fibrils of collagen can be crosslinked either by formation of ionic bonds with trivalent cations like chromium (Chvapil et al., 1973, Int. Rev. Connective Tissue Res. 6: 1-61) or aluminum (Gervais-Lugan et al., 1991, J. Biomed. Materials res. 25:1339-1346), using covalent crosslinkers (formaldehyde, glutaraldehyde, hexamethylenediisocyanate, polyepoxy compounds, carbodiimides) or using physical treatment (dry heat, exposure to ultraviolet, γ-irradiation, or pH changes) (Khor, 1997, Biomaterials 18:95-105).

Gelatin is one of the few materials that has been used successfully as a stabilizer in a vaccine (Sarkar et al., 2003, Vaccine 21:4728-4735; de Souza Lopes et al., 1988, J. Biologic. Standardization 16:71-76). Biodegradable nanoparticles of gelatin have been used to deliver drugs for the treatment of pulmonary diseases (Brzoska et al., 2004, Biochem. Biophys. Res. Comm. 318:562-570), to target T cells by conjugating specific antibodies to the surface of gelatin nanoparticles (Dinauer et al., 2005, Biomaterials 26:5898-5906; Balthasar et al., 2005, Biomaterials 26:2723-2732), and in photodynamic therapy preparations (Zhao et al., 2004, Biochim. Biophys. Acta 1670-113-120). Gelatin hydrogels have been tested as a new gene delivery system (Kasahara et al., 2003, J. Amer. Coll. Cardiol. 41:1056-1062) because of their positively charged nature and their biodegradability. The positively charged structure of gelatin is capable of encapsulating negatively charged nucleic acids, proteins and drugs. These gelatin-bound biomolecules were released when the gelatin gel gradually degraded. Further, it has been demonstrated that the infectivity of retroviruses can be preserved by freeze-drying when gelatin and sucrose are added (Levy and Fieldsteel, 1982, J. Virol. Meth. 5:165-171).

Borek et al. speculated that it is possible to provoke formation of antibodies that cross-react with a protein of the same animal species by immunization of the animal with a synthetic antigen (Borek et al., 1969, Biochim. Biophys. Acta 188:314-323). Several instances of anaphylactic shock that result following initial exposure to the antigen have been reported worldwide (Ring and Messmer, 1977, Lancet 1:466-469; Van Asperen et al., 1981, The Med. J. Australia 2:330-331; Aukrust et al., 1980, Allergy 35:581-587). The reaction of a seventeen year old who was vaccinated with measles, mumps, rubella (MMR) vaccine was attributed to the elicitation of an IgE antibody directed against the gelatin component of the vaccine (Keslo et al., 1993, J. Allergy and Clinical Immunol. 91:867-872). There are other reports citing the link between gelatin, as a heat stabilizer component of the vaccine, and anaphylaxis (Sakaguchi et al., 1996, J. Allergy and Clinical Immunol. 98:1058-1061; Sakaguchi et al., 1995, J. Allergy and Clinical Immunol. 96:563-656; Kumagai et al., 1997, J. Allergy and Clinical Immunol. 100:130-134; Sakaguchi and Inouye, 1998, Vaccine 16:68-69; Nakayama et al., 1999, J. Allergy and Clinical Immunol. 103:321-325; Sakaguchi et al., 1999, Immunology 96:286-290). These reports were further supported by the observation of a dramatic reduction in allergic responses when the formulation of the gelatin was altered (Nakayama and Aizawa, 2000, J. Allergy and Clinical Immunol. 106:591-592) or when the gelatin was removed from the vaccine entirely (Kuno-Sakai and Kimura, 2003, Biologicals 31:245-249). The majority of the reported cases of anaphylaxis originated in Japan and not in the U.S. (Pool et al., 2002, Pediatrics 110:71). It has been suggested that the hypersensitivity to gelatin based vaccines is caused by the strong association between gelatin allergy and HLA-DR9 (Kumagai et al., 2001, Vaccine 19:3273-3276, which is unique to the Asians population (Nakayama and Kumagai, 2004, Pediatrics 113:170-171). Pool et al. (2002, Pediatrics 110:71) also proposed that the addition of poorly hydrolyzed gelatin to diphtheria-tetanus-acellular pertussis (DTaP) vaccines in Japan may have contributed to a sensitization to gelatin in some children, resulting in increased risk of anaphylaxis on subsequent MMR vaccination (Nakayama et al., 1999, J. Allergy and Clinical Immunol. 103:321-325). The gelatin used in the vaccines manufactured in the US was found to be fully hydrolyzed.

Much research has been directed to the use of single walled nanotubes (SWNT) to transfer various macromolecules into mammalian cells. For example, such systems have been used in conjunction with small peptides (Pantarotto et al., 2004, Chemical Communications (Cambridge, UK: 16-17), nucleic acids (Lu et al., 2004, Nano Lett. 4:2473-2477) and proteins, for example, streptavidin (ShiKam et al., 2004, J. Amer. Chem. Soc. 126:6850-6851). Recently, Shikam et al. explored the destruction of cancer cells by functionalization of SWNT with a folate moiety (ShiKam et al., 2005, PNAS 102:11600-11605). This resulted in internalization of the SWNTs inside cells labeled with folate receptor tumor markers. Death of the tumor cells was achieved by irradiation of the cells with near infrared. Naguib et al. demonstrated that the surface of the carbon nanofiber structures could be tailored to have various biomedical applications simply by altering the post synthesis treatment (Naguib et al., 2005, Nanotechnology: 567-571). Similar results were reported by Salvador-Morales et al. (2006, Mol. Immunol. 43-193-201) in their work on protein adsorption on carbon nanotubes. Most importantly, functionalization of carbon nanotubes in order to enhance virus specific neutralizing antibody responses to peptides has been demonstrated by Pantarotto et al. (2003, Chem. Biol. 10:961-966). Using these technologies, the compound to be delivered is associated with the outside of the nanotube and the nanotube size then effects the delivery of the compound.

Despite all of the advances in vaccine technologies and in antiviral technologies in general, there remains a long-felt need in the art for viral vaccines, and especially influenza virus vaccines, that are safe and can be generated rapidly and with ease. These vaccines must be capable of eliciting a complete humoral and cellular immune response such that the vaccinated animal or human is fully protected against subsequent challenge by a virulent strain of virus. The present invention meets this need. Further, administration to an animal of a potentially lethal bioactive agent such as a live virus may have an adverse effect on others in the area if the agent should become aerosolized or spilled. The present invention solves this problem by providing compositions and methods for safe administration of such agents.

### BRIEF SUMMARY OF THE INVENTION

The invention includes a vaccine comprising a CD8+ T cell immunoprotective and/or antibody immunoprotective amount of virus, wherein the virus induces an immunoprotective CD8+ T cell and/or antibody response in an animal following administration of the virus to the animal by a route that does not cause disease in the animal.

Invention further includes a vaccine comprising a CD8+ T cell immunoprotective amount of virus, wherein the virus induces an immunoprotective CD8+ T cell response in an animal following administration of the virus to the animal by a route that does not cause disease in the animal.

In some aspects, the virus is a live virus, an attenuated virus or a killed virus.

In other aspects, the virus is a respiratory virus. In other aspects, the virus is selected from the group consisting of an orthomyxovirus, a paramyxovirus, a coronavirus, a picornavirus, respiratory syncytial virus, measles virus, adenovirus, a parvovirus, and adenovirus, a calicivirus, an astrovirus, Norwalk virus, an arenavirus, a flavivirus, a filovirus, a hantavirus, an alphavirus, a retrovirus and a lentivirus.

Preferably, the virus is an orthomyxovirus, more preferably, an influenza virus and even more preferably, the virus is influenza virus type A. When the virus is influenza virus type A, the virus has a hemagglutinin antigen (HA) selected from the group consisting of H1, H2, H3, H4, H5, H6, H7, H8, H9, H10, H11, H12, H13, H14, H15 and H16, a neuraminidase antigen (NA) selected from the group consisting of N1, N2, N3, N4, N5, N6, N7, N8 and N9. Even more preferably, the influenza virus type A has a HA:NA antigenic profile selected from the group consisting of H5N1, H9N2, H7N1, H7N2, H7N3, H7N7, H2N2, H1N1, H1N2 and H3N2.

In some embodiments, the vaccine comprises a low dose of the influenza virus type A. preferably, the low dose of influenza type A virus is from 0.001 to 5000 hamagglutination units (HAU) of virus, more preferably, from 0.005 to 500 HAU of virus and even more preferably, from 0.01 to 100 HAU of virus.

In some embodiments, the animal is a vertebrate, preferably a mammal and more preferably a human.

The vaccine of the invention may comprise a combination of viruses of two or more of member selected from the group consisting of a live virus, an attenuated virus, and a killed virus.

In preferred embodiments, the route of administration is a non-natural route, and more preferably is selected from the group consisting of subcutaneous, intradermal, intramuscular, mucosal and oral.

Also included in the invention is a kit comprising the vaccine of the invention.

The invention further relates to a vaccine comprising a CD8+ T cell immunoprotective and/or antibody immunoprotective amount of virus, wherein the virus induces an immunoprotective CD8+ T cell and/or antibody response in an animal following administration of the virus to the animal by a route that does not cause disease in the animal, and further wherein the virus is associated with an encapsulation vehicle.

In addition, the invention relates to a vaccine comprising a CD8+ T cell immunoprotective amount of virus, wherein the virus induces an immunoprotective CD8+ T cell response in an animal following administration of the virus to the animal by a route that does not cause disease in the animal, and further wherein the virus is associated with an encapsulation vehicle.

In a preferred embodiment, virus is encapsulated in the encapsulation vehicle, and may also be associated with a nanotube, a lipsome or a protein prior to being encapsulated in the encapsulation vehicle. In other embodiments, the encapsulation vehicle comprises one or more members selected from the group consisting of a gel, a liquid or a powder. Preferably, the encapsulation vehicle is loaded into a nanotube.

In certain embodiments, the encapsulation vehicle comprises a polymer and more preferably, is not toxic when administered to an animal. Preferably, the polymer is associated with the virus thereby delaying release of the virus into the surrounding environment.

In preferred embodiments, the polymer is a gel and may comprise collagen. The polymer may also be a hydrogel, and may preferably be selected from the group consisting of an alginate, gelatin, chitosan and hyaluronic acid, polyvinylpyrrolidone and carboxymethyl cellulose.

In other preferred embodiments, the gel comprises a combination of one or more of collagen, alginate, gelatin, chitosan, hyaluronic acid, polyvinylpyrrolidone and carboxymethyl cellulose.

Preferably, the gel is crosslinked and in addition, the gel may further comprise an additive. In a preferred embodiment, the additive is polyethylene glycol.

In other embodiments, the encapsulation vehicle comprises a microcapsule or a nanocapsule, or a nanotube. Preferably, the nanotube has a diameter of 500 nm or less.

In yet other embodiments, the encapsulation vehicle comprises a combination of one or more of a solution, a powder or a gel.

The encapsulation vehicle preferably comprises a virus as described elsewhere herein.

Also included in the invention is a device for delivery of a vaccine to an animal, the device comprising (a) a CD8+ T cell immunoprotective and/or antibody amount of virus, wherein the virus induces an immunoprotective CD8+ T cell and/or antibody response in an animal following administration of the virus to the animal by a non-natural route, (b) a delivery device for delivering the vaccine to the animal.

Further included is a device for delivery of a vaccine to an animal, the device comprising (a) a CD8+ T cell immunoprotective amount of virus, wherein the virus induces an immunoprotective CD8+ T cell response in an animal following administration of the virus to the animal by a non-natural route, (b) a delivery device for delivering the vaccine to the animal.

In one embodiment, the delivery device comprises a hollow tube, and preferably, the hollow tube has a tapered end. In some embodiments, the delivery device comprises a needle. In other embodiments, the hollow tube is optionally attached to a plunging device, where preferably, the plunging device is a syringe, a gene gun, a catheter, a patch, an inhaler, or a mucosal applicator.

The device preferably comprises an encapsulation vehicle and a virus as described elsewhere herein. Preferably, the virus is encapsulated in the encapsulation vehicle and more preferably, is associated with a nanotube, a lipsome or a protein prior to being encapsulated in the encapsulation vehicle.

There is further included in the invention a method of making a vaccine comprising a CD8+ T cell immunoprotective and/or antibody immunoprotective amount of virus. The method comprises combining a CD8+ T cell and/or antibody immunoprotective amount of a virus with an encapsulation vehicle, thereby making the vaccine.

Also included is a method of making a vaccine comprising a CD8+ T cell immunoprotective amount of virus where the method comprises combining an immunoprotective amount of a virus with an encapsulation vehicle, thereby making the vaccine.

The invention also includes a method of eliciting a CD8+ T cell immunoprotective and/or antibody immune response in an animal. The method comprises administering to the animal a vaccine comprising a CD8+ T cell and/or antibody immunoprotective amount of virus, whereby a CD8+ T cell and/or antibody immune response is elicited in the mammal.

In addition, there is included a method of eliciting a CD8+ T cell immune response in an animal. The method comprises administering to the animal a vaccine comprising a CD8+ T cell immunoprotective amount of virus, whereby a CD8+ T cell immune response is elicited in the animal.

In these and other methods, preferably the animal is a mammal and more preferably, the mammal is a human.

Also included in the invention is a method of protecting an animal against infection by a virus. The comprises administering to the animal a vaccine comprising a CD8+ T cell immunoprotective and/or antibody immunoprotective amount of the virus, whereby a CD8+ T cell and/or antibody immune response is elicited in the animal thereby protecting the animal against the infection.

In addition, there is provided a method of protecting an animal against infection by a virus where the method comprises administering to the animal a vaccine comprising a CD8+ T cell immunoprotective amount of the virus, whereby a CD8+ T cell immune response is elicited in the animal thereby protecting the animal against the infection.

Also included is a method of preventing a virus infection in an animal where the method comprises administering to the animal a vaccine comprising a CD8+ T cell immunoprotective and/or antibody immunoprotective amount of the virus, whereby a CD8+ T cell and/or antibody immune response is elicited in the animal thereby preventing a virus infection in the animal.

In addition, there is provided a method of preventing a virus infection in an animal. The method comprises administering to the animal a vaccine comprising a CD8+ T cell immunoprotective amount of the virus, whereby a CD8+ T cell immune response is elicited in the animal thereby preventing a virus infection in the animal.

Further included is a method of treating a virus infection in an animal. The method comprises administering to the animal a vaccine comprising a CD8+ T cell immunoprotective and/or antibody immunoprotective amount of the virus, whereby a CD8+ T cell and/or antibody immune response is elicited in the animal thereby treating the animal.

Also included is a method of treating a virus infection in an animal where the method comprises administering to the animal a vaccine comprising a CD8+ T cell immunoprotective and/or antibody immunoprotective amount of the virus, whereby a CD8+ T cell and/or antibody immune response is elicited in the animal thereby treating the animal.

The invention includes a composition comprising a CD8+ T cell immunoprotective and/or antibody immunoprotective amount of a bioactive agent, wherein the bioactive agent induces an immunoprotective CD8+ T cell and/or antibody response in an animal following administration of the bioactive agent to the animal by a route that does not cause disease in the animal.

The invention also includes composition comprising a CD8+ T cell immunoprotective amount of bioactive agent, wherein the bioactive agent induces an immunoprotective CD8+ T cell response in an animal following administration of the bioactive agent to the animal by a route that does not cause disease in the animal.

In preferred embodiments, the route is a non-natural route and may be selected from the group consisting of subcutaneous, intradermal, intramuscular, mucosal and oral. The bioactive agent is encapsulated in the encapsulation vehicle and preferably, the bioactive agent is associated with a nanotube, a lipsome or a protein prior to being encapsulated in the encapsulation vehicle. The the encapsulation vehicle comprises at least one member selected from the group consisting of a gel, a liquid or a powder and preferably, the encapsulation vehicle is loaded into a nanotube. The encapsulation vehicle may also comprise a polymer and preferably, the polymer is not toxic when administered to an animal. The polymer may be associated with the bioactive agent thereby delaying release of the bioactive agent into the surrounding environment. Preferably, the polymer is a gel and also preferably, the bioactive agent is selected from the group consisting of a microorganism, and a protein.

Also included is a method of enhancing safety when administering a bioactive agent to an animal. The method comprises administering a composition comprising a CD8+ T cell immunoprotective and/or antibody immunoprotective amount of a bioactive agent to the animal, wherein the bioactive agent induces an immunoprotective CD8+ T cell and/or antibody response in the animal following administration of the bioactive agent by a route that does not cause disease in the animal and further wherein the bioactive agent is encapsulated in an encapsulation vehicle.

Further included is a method of enhancing safety when administering a bioactive agent to an animal. The method comprises administering to the animal a composition comprising a CD8+ T cell immunoprotective amount of bioactive agent, wherein the bioactive agent induces an immunoprotective CD8+ T cell response in the animal following administration of the bioactive agent by a route that does not cause disease in the animal and further wherein the bioactive agent is encapsulated in an encapsulation vehicle.

In preferred embodiments, the bioactive agent is selected from the group consisting of a microorganism and a protein.

In addition, there is included in the invention a composition comprising a biologically effective amount of a bioactive agent, wherein the bioactive agent induces a desired response in an animal while reducing risk in an animal following administration of the bioactive agent to the animal by a route that does not cause disease in the animal.

Further included is a method of enhancing safety when administering a bioactive agent to an animal. The method comprises administering to an animal a composition comprising an amount of a bioactive agent that induces a desired response while reducing risk in an animal, wherein the route of administration of the bioactive agent is a route that does not cause disease in the animal, and further wherein the bioactive agent is encapsulated in an encapsulation vehicle thereby enhancing safety when administering the bioactive agent.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiment(s) which are presently preferred. It should be understood, however, that invention is not limited to the precise arrangements and instrumentalities shown. In the drawings:

FIG. 1 is a bar graph depicting the effects of four different routes of administration of influenza virus on the secondary virus-specific CD8+ T cell response in C57Bl/6J mice. Mice were primed with 100 hemagglutination units (HAU) of PR8 influenza by intraperitoneal (IP), intramuscular (IM), intradermal (ID) or subcutaneous (SubQ) injection routes. Tissues were harvested seven days after rechallenge with X31 influenza A virus and the presence of virus-specific CD8+ T cells was assessed in preparations of lung tissue using an MHC-class 1 tetramer loaded with immunodominant Influenza type A virus nuclear protein NP₃₆₆₋₃₇₄ (ASNENMETM (SEQ ID NO:1)).

FIG. 2, comprising FIGS. 2A and 2B, provide the results of a dose response study in mice. In this study, the secondary virus-specific CD8+ T cell response was assessed in C57Bl/6J mice primed with various doses of PR8 influenza virus administered by either IP or ID injection routes. Tissues were harvested seven days after intranasal rechallenge with X31 influenza A virus. Virus-specific CD8+ T cells were detected in lung preparations of lung tissue using an MHC class I tetramer loaded with the immunodominant Influenza type A virus nuclear protein NP₃₆₆₋₃₇₄ (ASNENMETM (SEQ ID NO:1)) or IFNγ intracellular stain. FIG. 2A depicts representative FACS plots of virus-specific CD8+ T cells. FIG. 2B depicts dose response curves of virus-specific CD8+ T cells and IFNγ producing CD8+ T cells. Points are the mean+/−SEM for three animals per group (*p<0.05).

FIG. 3, comprising FIGS. 3A and 3B, is a series of graphs depicting virus-specific CD8+ T cell responses to 1 HAU of live influenza virus delivered IP, SQ or ID. Secondary virus-specific CD8+ T cell response in C57Bl/6J mice primed with 1 HAU of PR8 influenza virus by different infection routes: IM, SQ or ID. Lungs were harvested at seven days after intranasal rechallenge with X31 influenza virus and virus-specific CD8+ T cells were detected using MHC class I tetrameric complexes loaded with the immunodominant peptide epitope derived from the viral nucleoprotein: NP366-374 (ASNENMETM SEQ ID NO:1). Percentage NP₃₆₆-specific CD8+ T cells out of total CD8+ T cells (A) and total numbers of NP₃₆₆-specific CD8+ T cells (B) were calculated in each of the three immunization conditions. Horizontal line depict mean value.

FIG. 4 is a graph depicting the safety of live influenza vaccine administered SQ. Wild type C57Bl/6J (white diamonds) or immunodeficient Rag−/−γc−/− mice (white circles) were SQ immunized with 100 HAU of live PR8 influenza virus. As a control, a group of C57Bl/6J mice (black diamonds) were infected IN with 1 HAU of PR8 influenza virus. The weight of the mice was recorded over the next seventeen days and the percentage weight loss was plotted against the number of days following infection.

FIG. 5 is a graph depicting the fact that influenza A virus administered subcutaneously is safe and does not cause disease. Wild type C57BL/6 mice were administered influenza virus intranasally at a low dose (0.1 HAU) of PR8 (black squares) or the London strain (black triangles). Immunodeficient Rag−/−γc−/− mice were injected subcutaneously with a high dose (10 HAU) of either PR8 (open diamonds) or London virus (black circles). The weight of the mice was monitored over 30 days post-inoculation. The average weight loss post-inoculation is shown. Moribund mice were euthanized at 30% weight loss (+). For all groups, n=5.

FIG. 6, comprising FIGS. 6A, 6B and 6C, is a series of flow cytometry images depicting the fact that live virus in gelatin gel administered SQ to efficiently stimulates a CD8+ T cell response in the mice. Lungs (FIG. 6A) and spleens (FIG. 6B) from un-manipulated mice or from mice immunized with gelatin alone, gelatin and virus (10 HAU) or virus alone (10 HAU) for thirty days, were analyzed seven days after intranasal rechallenge with X31 influenza virus. Single cell suspensions were stained with anti-CD8 antibodies and MHC class I/NP366-374 tetrameric complexes and were analyzed by flow-cytometry. Percentage of CD8+ T cells within total lymphocytes is indicated outside the gate whereas the percentage NP₃₆₆-specific CD8+ T cells within total CD8+ T cells is indicated inside the box. (FIG. 6C) Splenocytes from the indicated mice were in vitro stimulated with NP366-374 peptide for six hours. IFNγ production by CD8+ T cells was assessed by intracellular staining with anti-IFNγ antibodies and flow-cytometry analysis (percentage IFNγ+CD8+ T cells indicated in the quadrant).

FIG. 7, comprising FIG. 7a, FIG. 7b, and FIG. 7c and FIG. 7d, is a series of images of electron micrographs depicting collagen polymers having different pore sizes that were produced by varying polymer concentration and crosslinker content. SEM micrographs of freeze-dried collagen gels fabricated inside a needle containing: 6 mg/ml of collagen (FIG. 7a); 10 mg/ml of collagen (FIG. 7b); collagen gel containing 10 mg/ml collagen (wet-mode ESEM) (FIG. 7c); and freeze-dried collagen:PEG hydrogel at a ratio of 1:4 (FIG. 7d).

FIG. 8, comprising FIGS. 8A and 8B, is a graph (FIG. 8A) and a table (FIG. 8B) depicting the effect of polymer properties and Ca²⁺ concentration (i.e., vehicle properties) on ejection times.

FIG. 9, comprising FIGS. 9a, and 9b, is a series of graphs depicting the fact that polymer properties control nanoparticle release rate. QDot (20 nm size) were released from alginate polymers of low viscosity (FIG. 9a) and high viscosity (FIG. 9b). Low viscosity polymer rapidly released QDots whereas high viscosity polymer released QDots at a much slower rate.

FIG. 10 is a graph depicting the fact that live PR8 virus delivered in alginate gels potently stimulates CD8+ T cell responses in mice. Large numbers of pulmonary NP₃₆₆ specific CD8+ T cells were elicited in animals that were inoculated subcutaneously with live virus and then challenged with virulent virus. Lungs from unmanipulated mice or from mice that were inoculated subcutaneously with PR8 live virus alone, alginate alone, or PR8 live virus encapsulated in alginate, were analyzed at seven days after intranasal rechallenge with X31 virus. Single cell suspensions were stained with anti-CD8 antibodies and MHC class I/NP₃₆₆₋₃₇₄ peptide and were analyzed by flow cytometry. The values shown represent the average values obtained from two mice per group.

FIG. 11, is a graph depicting the fact that Live PR8 virus delivered in alginate gels efficiently stimulates production of influenza virus-specific antibodies. Anti-PR8 antibodies present in the serum of C57BL/6 mice immunized with PR8 virus alone or encapsulated in alginate gel, were detected by ELISA using PR8 virus as the capturing antigen. The 1/270 initial serum dilution was further diluted in three fold serial dilutions and was added to plate-bound PR8 virus. Uninfected animals exhibited no antibody response to PR8 virus.

FIG. 12 is a graph depicting the fact that vaccination of mice with live virus encapsulated in alginate gels elicits neutralizing antibodies in the serum of the animals. Serum from mice infected with live virus encapsulated in alginate was serially diluted (1/2 serial dilutions) and was tested for the ability to inhibit chicken red blood cell hemagglutination by 2 HAU of PR8 virus. Serum from animals that were not inoculated with virus did not inhibit hemagglutination.

FIG. 13 is an image of a scanning electron micrograph of a carbon nanotube synthesized by template assisted pyrolysis of ethylene at 670° C. The diameter of the tube was determined by the pore diameter of the template, which was 250 nm in the present case. The thickness of the nanotube wall is approximately 20 nm.

FIG. 14 is a schematic illustration of carbon nanotube synthesis by a chemical vapor deposition process.

FIG. 15, comprising FIGS. 15a and 15b, is a series of confocal images of micrographs depicting the fact that nanotubes can be loaded with alginate gels that contain QDots. Confocal images of carbon nanotubes filled with sodium alginate and quantum dots are shown. Nanotubes were mixed with alginate gel that contained 50 nm QDots and then underwent the loading procedure. The presence of fluorescence inside the nanotubes is evidence of loading. Arrows point to individual tubes.

FIG. 16, comprising FIGS. 16a and 16b, is a series of images of QDots in alginate gel loaded into nanotubes. SEM images of carbon nanotubes containing sodium alginate and QDots are shown. Gel and QDots can be clearly seen inside the tubes. The scale bars on both images measures 2 μm.

FIG. 17, comprising FIGS. 17a and 17b, is a series of confocal images of nanotubes mixed with QDots but that did not load. Nanotubes were mixed with alginate gel that contained 50 nm QDots but were not subjected to the loading procedure. Fluorescence of the QDots is evident in the background, but not in the tubes. Arrows point to individual tubes.

FIG. 18, comprising FIGS. 18a and 18b, is a series of SEM images depicting that nanotubes can be fragmented by sonnication. FIG. 18a-before sonnication; FIG. 18b-after sonnication at (×10,000 magnification) at 1.36 MHz, 30 seconds. Before sonnication, the tubes were 10-20 μm long. After sonnication, the tubes were <1 μm long.

### DETAILED DESCRIPTION OF THE INVENTION

The invention relates to the discovery of compositions and their use in novel vaccine strategies for protection of animals, preferably vertebrate animals, preferably mammals, and more preferably humans, against influenza virus infection. However, the invention should not be construed to be limited solely to vaccine strategies for protection of an animal against influenza virus infection, but rather should be construed to include administration of any bioactive agent which, if administered by certain routes may aerosolize or otherwise create exposure and pose a risk of harm to other vertebrate animals. The invention further includes vaccine strategies that confer protection against other viral infections, including, but not limited to, other RNA viruses that infect or enter the host via the respiratory or gasterointestinal tract of animals, and even in some instances, DNA viruses that infect or enter the host via the respiratory or gasterointestinal tract of animals. This is because as more fully described elsewhere herein, the vaccine strategy of the present invention relates to the discovery that administration of low doses of live virus to a vertebrate animal by different routes of administration than those presently in use for routine vaccination or that of natural entry of the virus into an animal, either alone or when combined with novel formulations and delivery vehicles, induces a potent immune response comprising CD4+ and CD8+ T cells and/or antibodies, that is critical for effective protection of the animal against subsequent challenge by infectious virus. For example, it known that administration of influenza virus to an animal by a route that is different from that of the natural infection, generally does not cause disease in the animal. However, current killed influenza vaccines that are administered intramuscularly to an animal elicit only a humoral and only a weak or no T cell immune response. Attenuated influenza virus vaccines that are administered intranasally (i.e., the natural route) elicit only a weak CD8+ T cell immune response. Thus, current vaccines protect approximately only 30% of the target population. The invention includes the finding that administration of a low dose live influenza virus vaccine subcutaneously or intradermally, induces a potent CD8+ T cell response and is therefore superior to current vaccines. In other words, the invention includes the administration of a bioactive agent to a vertebrate animal by a route that is not the natural route of entry of that bioactive agent into the animal, where a protective immune response is elicited in the animal that then protects the animal against disease upon subsequent challenge with the bioactive agent. The invention further includes the encapsulation of the bioactive agent in a material that reduces substantially the ability of the material to aerosolize or create other exposure risks, thereby rendering the administration of the bioactive agent more safe than in the absence of the material. These and other aspects of the invention will become apparent following a reading of the disclosure provided herein.

Definitions:

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, to “alleviate” a disease, disorder or condition means reducing the severity of one or more symptoms of the disease, disorder or condition.

As used herein, to “treat” means reducing the frequency with which symptoms of disease are experienced by an animal, preferably a vertebrate, preferably a mammal, more preferably a human.

By the term “applicator” as the term is used herein, is meant any device including, but not limited to, a needle, a catheter, a hypodermic syringe, a gene gun, a patch, a nanotube, a mucosal applicator, or any combination thereof, for administering the composition of the invention to a vertebrate.

By the term “bioactive agent” as used herein, is meant any agent that when administered to a vertebrate, causes an effect on the vertebrate. The effect caused may be beneficial or adverse to the vertebrate when the agent is administered to the vertebrate. Examples of bioactive agents include without limitation, live virus, attenuated or killed virus, inactivated virus, microorganisms that are live, attenuated or killed, peptide, protein, nucleic acid, or small organic or inorganic chemical, which can be administered as a vaccine, immunogen, drug or other therapeutic to a vertebrate, preferably a human.

As used herein, an “effective amount” of bioactive agent, e.g., a vaccine or other composition, means any amount that elicits a desired response. In the case of a vaccine, this term means any amount of the bioactive agent that when administered to a vertebrate elicits a CD8+ T cell and/or an antibody immune response directed against an antigen in the bioactive agent in the vertebrate.

As used herein, a “CD8+ T cell and/or antibody immunoprotective amount of bioactive agent” means an amount of bioactive agent that when administered to a vertebrate, elicits a CD8+ T cell response and/or antibody response directed against the bioactive agent, whereby, when the vertebrate is challenged with the bioactive agent through a route that would have an adverse effect on the vertebrate in the ordinary course, the vertebrate exhibits fewer or less serious symptoms of disease caused by the challenging bioactive agent than a second otherwise identical vertebrate similarly challenged, but that was not administered a CD8+ T cell and/or antibody immunoprotective amount of the bioactive agent.

As used herein, a “CD8+ T cell immunoprotective amount of bioactive agent” means an amount of bioactive agent that when administered to a vertebrate, elicits a CD8+ T cell response directed against the bioactive agent, whereby, when the vertebrate is challenged with the bioactive agent through a route that would have an adverse effect on the vertebrate in the ordinary course, the vertebrate exhibits fewer or less serious symptoms of disease caused by the challenging bioactive agent than a second otherwise identical vertebrate similarly challenged, but that was not administered a CD8+ T cell immunoprotective amount of the bioactive agent.

As used herein, the terms “gene” and “recombinant gene” refer to nucleic acid molecules comprising, at a minimum, an open reading frame encoding a polypeptide.

As used herein, an “instructional material” includes a publication, a recording, a diagram, or any other medium of expression which can be used to communicate the usefulness of the bioactive agent, vaccine or other composition of the invention in a kit for effecting alleviation of the various diseases or disorders recited herein. Optionally, or alternately, the instructional material may describe one or more methods of alleviation the diseases or disorders in a cell or a tissue of a vertebrate. The instructional material of the kit of the invention may, for example, be affixed to a container which contains the bioactive agent, vaccine or other composition of the invention or be shipped together with a container which contains the bioactive agent, vaccine or composition. Alternatively, the instructional material may be shipped separately from the container with the intention that the instructional material and the compound be used cooperatively by the recipient.

By the term “specifically binds,” as used herein, is meant a compound, e.g., a protein, a nucleic acid, an antibody, and the like, which recognizes and binds a specific molecule, but does not substantially recognize or bind other molecules in a sample.

As used herein, the term “transgene” means an exogenous nucleic acid sequence which exogenous nucleic acid is encoded by a transgenic cell or mammal.

By the term “live” as used herein to refer to a virus, is meant that the virus is capable of infecting and replicating in a host cells and of causing disease in an animal.

This is in contrast to the term “attenuated” as used herein to refer to a virus, by which is meant a virus that is capable of infecting a host cell, but has either significantly diminished or no capacity to cause disease in an animal.

The term “killed” virus as used herein to refer to a virus, is a virus that is incapable of infecting and replicating in a host cell and is also largely incapable of causing disease in an animal.

By the term “vaccine” as used herein is meant an antigen, i.e., a bioactive agent, preferably a virus or other microorganism or protein, that elicits an immune response in a vertebrate to which the vaccine has been administered. Preferably, the immune response confers some beneficial, protective effect to the vertebrate as against a subsequent challenge with the same or a similar bioactive agent. More preferably, the immune response prevents the onset of or ameliorates at least one symptom of a disease associated with the bioactive agent, or reduces the severity of at least one symptom of a disease associated with the bioactive agent upon subsequent challenge. Even more preferably, the immune response prevents the onset of or ameliorates more than one symptom of a disease associated with the bioactive agent upon subsequent challenge.

By the term “method or route that does not cause disease” is meant administering the bioactive agent in a manner that presents the agent to the organism in a way that is different from the mechanism or point of entry by which the agent would naturally be hazardous, toxic or infect the organism. By way of a non-limiting example, the point of entry of influenza virus during natural infection of a human is through through the respiratory tract as an unencapsulated virus. In this context, the “method or route that does not cause disease” is injection of the virus, preferably subcutaneously or intradermally, wherein the virus is encapsulated in an encapsulation composition.

By the term “non-natural route” as used herein is meant the point of entry of a virus in the body of an animal that is not a point of entry for the virus during natural infection of the animal by the virus. By way of example, the point of entry of influenza virus during natural infection of a human is the respiratory tract. Subcutaneous or intradermal routes of entry are therefore non-natural routes for entry of influenza virus.

By “natural route of infection” is meant the route by which the virus infects an animal during natural spread of the virus.

By “natural route of entry of a bioactive agent” is meant the route by which exposure of the animal to the bioactive agent would normally cause symptoms of disease associated therewith.

By the term “low dose” of virus is meant an amount of virus that is sufficient to elicit a protective CD8+ T cell and/or antibody response in a vertebrate in which the virus has been administered. The skilled practitioner will know the exact amount of virus to be administered in each situation, and the amount will vary depending on any one or more of a number of factors, including but not limited to, the virulence of the particular virus used, the age and overall health of the animal to which the virus is administered, the formulation of the virus, and even the device used for administration of the virus. In the case of influenza virus, a low dose may range from about 0.0001 hemagglutination units (HAU) to about 5000 HAU. Preferably, a low dose may range from about 0.0005 to about 500 HAU, more preferably, from about 0.001 to about 100 HAU and even more preferably, from about 0.05 to about 10 HAU and any and all whole or partial integers therebetween

By the term “respiratory virus” as used herein is meant a virus that upon infection of an animal, primarily uses the respiratory tract as a point of entry, and/or primarily targets the respiratory tract and causes respiratory disease in the animal.

By the term “enteric virus” as used herein is meant a virus that upon infection of a vertebrate, uses the gastrointestinal tract as a point of entry and/or primarily targets the gastrointestinal tract and causes gastrointestinal disease in the vertebrate.

“Subcutaneous” refers to the region of fatty tissue that lies between the dermis layer of the skin and the muscle tissue below.

“Intradermal” refers to the dermis layer which lies between the epidermis and the subcutaneous fat layer below. Intradermal sites contain large numbers of antigen presenting cells and provide faster release into lymphatics compared to subcutaneous sites. This may result in differences in type and magnitude of immune response, antigen/bioactive agent clearance, and required dose between intradermal and subcutaneous injection sites.

By the terms “vaccine unit” or “unit of vaccine” as used herein is meant an amount of vaccine that when administered to a vertebrate, initiates the elicitation of a protective immune response in the vertebrate. The vaccine unit may initiate the elicitation of a completely protective immune response in the vertebrate, or may initiate an incomplete response whereby additional vaccine units would be required for a complete response.

By the term “encapsulation vehicle” as used herein is meant a composition for administration of a bioactive agent, a vaccine or other composition, to a vertebrate where the composition coats, surrounds, encompasses, or otherwise is associated with the agent, such that the agent comprises additional material from that present in its non-encapsulated state.

By the term “biocompatible polymer” is meant a polymer that when administered to an animal does not induce a reaction that is generally adverse to the animal. This term is used synonomously herein with the term “non-toxic.”

By the term “microcapsule” as used herein is meant a vehicle that surrounds or is otherwise associated with a bioactive agent and provides a barrier between the agent and the environment. Dimensions of microcapsules are of the order of about one to several hundred microns and any and all whole or partial integers therebetween. Shapes of microcapsules may vary and include but are not limited to spherical, ellipsoidal and polygonal formations. The form of encapsulation can vary from being evenly spaced throughout a matrix, often referred to as a microsphere, to being confined in one part, for example in case of a hollow microcapsule filled with bioactive agent.

By the term “nanocapsule” as used herein is meant a microcapsule-like structure where the dimensions are in the range of about 1 nm to 1 micron and any and all whole or partial integers therebetween.

By the term “nanotube” as used herein is meant a structure having a length to width ratio of larger than 1, having a cross section of any shape (circular, ellipsoidal, rectangular, polygonal or other), wherein one dimension is of the order of 100 nm or less but can measure up to 1 micron, and any and all whole or partial integers therebetween.

By the term “delivery device” as used herein is meant a device that can penetrate at least outermost layer of the skin of a vertebrate and deliver a bioactive agent to an internal tissue of the vertebrate. Alternatively, the delivery device can deliver a bioactive agent to a mucosal tissue in a vertebrate. A non-limiting example of a delivery device are needles, syringes, catheters, gene guns, nanotubes, patches, mucosal applicators and the like.

As used herein, a “safe delivery vehicle or device” is a means for delivering a potentially hazardous bioactive agent to a vertebrate, where if the vertebrate was exposed to the bioactive agent in a non-safe mode, generally as an aerosol or free powder form, the bioactive agent would have an adverse effect on the vertebrate.

Description:

I. Viruses and Other Bioactive Agents:

The invention is based on the discovery that low dose subcutaneous or intradermal administration of live influenza virus in mice induces a potent CD4+ and CD8+ T cell response and an antibody response in the mice that protects them against subsequent challenge by infectious influenza virus administered intranasally. The invention is further based on the discovery that the risk associated with the administration of a bioactive agent that has the potential to form a hazardous aerosol can be minimized if the bioactive agent is encapsulated in a material that prevents aerosolization of the bioactive agent.

The invention should not be construed to be limited solely to the use of vaccines that are directed against influenza virus, but rather should be construed to include development of vaccines against other viruses, particularly respiratory or enteric viruses. In addition, the invention should be construed to include the administration of a bioactive agent to a vertebrate animal, where the agent is potentially hazardous in aerosol or powder form. Further, the invention should be construed to include vaccines that are directed, not only against viruses, but against other microorganisms, including, but not limited to bacteria. The invention should further be construed to include the administration of vaccines that are directed against other molecules, compounds or structures comprised of, but not limited to proteins or lipids.

As described in more detail elsewhere herein, the present invention includes a vaccine that is capable of inducing a protective CD4+ T cell response, or a protective CD8+ T cell response, or an antibody response against a given bioactive agent, or a combination of two or more of each response.

Other viruses that are included in the vaccines of the present invention are those that similarly rely on a T cell response, i.e., a CD4+ and/or a CD8+ T cell response, and/or an antibody response for protection therefrom. Such viruses include, but are not limited to, RNA viruses, RNA viruses that cause respiratory infection, and in some instances, DNA viruses. Non-limiting examples of these viruses include orthomyxoviruses, paramyxoviruses, respiratory syncytial virus, coronaviruses, measles virus, adenovirus, enteroviruses (including without limitation, picorna viruses such as poliovirus, coxsackieviruses, echoviruses, parvoviruses, rotaviruses, caliciviruses, astroviruses, Noroviruses, Norwalk virus, arboviruses and arenaviruses, e.g., flaviviruses, filoviruses, hantaviruses, alphaviruses, retroviruses or lentiviruses, and the like.

Thus, it should be noted that although influenza virus is exemplified throughout the present disclosure, the invention must be construed to include these additional viruses and other microorganisms as an integral part of the present disclosure as well as other potentially hazardous bioactive agents. Once armed with the present invention, it is well within the skill of the artisan to develop additional viral compositions and vaccines having the property of being capable of inducing a protective CD4+ T cell, and/or CD8+ T cell immune response, and/or an antibody response that is beneficial to the immunized individual upon subsequent challenge by infectious virus. It is further well within the skill of the artisan to develop additional vehicle/agent combinations that facilitate safe administration of agents to animals and humans.

Given this, the disclosure here that focuses primarily on influenza virus is for the purposes of clarity only and should be construed to be generally applicable to other bioactive agents, microorganisms and viruses whose pathogenesis, replication and/or infectious disease cycles are known and can be manipulated to generate effective vaccines following the general procedures disclosed herein in conjunction with those in the art. For a review of these procedures see Fields Virology by Bernard Fields, Editor, David KnipeLippincott Williams & Wilkins; 3nd edition (1996).

There is a plethora of information in the art that teaches the growth and assessment of various viruses in various cell or other systems, for example in the case of influenza virus, eggs are used to generate virus for vaccine production. Each virus has its own system whereby large amounts are produced, and these systems are well known and are readily available to the skilled artisan. When large quantities of live virus are generated for use in a vaccine, the virus produced must be capable of infecting host cells and replicating therein. Further, in the case of a live virus vaccine, the ability of the virus to cause disease in an infected host, when the infection is by the natural route, is assessed using methodology readily available to the skilled artisan. Viruses that can be replicated, isolated, are capable of infecting cells, and that cause disease in an animal when the natural route of infection is used, are candidates for use in the live virus vaccines of the present invention. Viruses that can be replicated and isolated as attenuated viruses such that they are capable of infecting cells, but do not cause overt disease in an animal when the natural route of infection is used, are candidates for use in the attenuated virus vaccines of the present invention. Viruses that can be replicated, isolated, and then are killed such that they are not capable of infecting cells and do not cause disease in an animal when the natural route of infection is used, are candidates for use in the killed virus vaccines of the present invention. Finally, other microorganisms that can cause disease following entry by natural routes either as wild type, attennuated or killed organisms and viruses, are candidates for use in the vaccines of the present invention.

When the virus is used in a vaccine, the virus is typically administered to an animal, preferably a mammal, and more preferably, a human. However, the invention should be construed to include administration of the virus, other microorganism, or the bioactive agent, to a variety of animals, including, but not limited to, cats, dogs, horses, cows, cattle, sheep, goats, birds such as chickens, ducks, geese, and fish.

Two types of influenza virus vaccines are presently in use globally. These are (i) intramuscular injection of killed virus and (ii) intranasal administration of attenuated virus. The administration of either vaccine induces a strong antibody response against the virus that is protective against subsequent viral infection.

There are several disadvantages associated with either vaccine and these are now documented herein. (a) The induced antibody response does not, in and of itself, provide sufficient protection against subsequent infection by virus. A protective CD8+ T cell response directed against the virus is also required. Such a CD8+ T cell response can be induced in healthy individuals having antibody against the virus when infected by the prevailing infectious strain, but may not be induced in immunocompromised individuals or in the very young or old. (b) The antibody response induced following vaccine administration is highly specific for the strain of virus that is used as the immunogen. Thus, it becomes necessary to identify, prepare and administer new vaccines yearly in order to immunize the population. If the virus strain used in the vaccine turns out to be a different strain than the prevailing infecting strain in any given year, then the morbidity and mortality of influenza virus infection is increased since most of the population will not be protected against the prevailing strain. (c) The amount of virus that is required to efficiently induce a protective antibody response against influenza virus in any given year is vast, production is complex and all too frequently, not enough vaccine can be generated in the time required to sufficiently immunize a substantial portion of the population.

The vaccine of the present invention comprises a low dose of live infectious virus that is administered to a vertebrate by an intradermal or subcutaneous route. Subcutaneous refers to the region of fatty tissue that lies between the dermis layer of the skin and the muscle tissue below. Intradermal refers to the dermis layer which lies between the epidermis and the subcutaneous fat layer below. Intradermal sites contain large numbers of antigen presenting cells and provide faster release into the lymphatics compared to subcutaneous sites. This may result in differences in the type and magnitude of immune response, antigen/bioactive agent clearance, and required dose between intradermal and subcutaneous injection sites. As will be apparent upon a reading of the present disclosure, there are several advantages to using the present vaccine over those that are currently in use. First, the vaccine of the present invention confers a protective immune response to all recipients because the vaccine of the present invention induces, in addition to an antibody response, a CD4+ and a CD8+ T cell response in recipients, something that is critical for a more complete protection against subsequent virus infection. Second, the specificity of the vaccine for individual strains of virus is less critical in that the protective CD4+ and CD8+ T cell responses induced by the present vaccine is less specific for each individual virus serotype as many of the internal segments/genes of influenza virus strains share antigenic T cell epitopes. Third, low doses of virus are sufficient and therefore the difficulties associated with the generation of large amounts of any particular virus are diminished. Once the prevailing infectious strain is isolated, large amounts of vaccine can be rapidly produced and therefore large segments of the susceptible population can be immunized more quickly than is presently possible. Fourth, a single injection of live virus elicits an immune response which can be further boostered by additional immunization. Current vaccines require multiple vaccinations to elicit protective immune responses.

The virus that is used as a vaccine in the present invention is preferably a “live” virus. However, attenuated viruses and killed viruses, or combinations of any or all of these viruses, or any bioactive agent eliciting a CD8+ cell and/or antibody response, are also contemplated by the present invention. In the case of influenza virus, the type of virus to be used in a vaccine is preferably influenza virus type A, although other influenza viruses that are known, or are as yet unknown, are also included in the invention. As discussed elsewhere herein, there presently exists a number of different serotypes of influenza virus type A, and their ability to cause disease and induce immunity in humans and other animals is governed in large part by the type of HA and NA antigens in the envelope of the virus. The present invention should be construed to include any and all viruses having any and all combinations of HA and NA antigens in the viral envelope, irrespective of whether these virus strains are produced during natural infection of a host, are produced by reassortment of HA and NA antigens as a result of infection of different species, or are produced by recombinant means where the antigenic make up of the virus is either specifically designed or is generated by random recombination as is possible using ordinary molecular biology techniques. Preferably, the influenza virus useful in the invention is one that is capable of eliciting a broad spectrum CD8+ T cell and/or antibody response in a vertebrate. Most preferably, the virus consists of, but is not limited to, those of potential pandemic strains of influenza virus (for example H5N1, H9N2, H7N, H7N2, H7N3 or H7N7), past pandemics (for example H2N2 or H1N1), or non-pandemic viruses (for example H1N1, H1N2 or H3N2).

As noted elsewhere herein, the invention should not be construed to be limited solely to the use of a live virus as a vaccine. Attenuated viruses, as well as killed viruses, or combinations of attenuated virus, killed and live viruses, are also contemplated as being useful and encompassed by the present invention. Other live, killed or attenuated microorganisms are also contemplated as being useful and encompassed by the present invention. The skilled practitioner will understand, based on the disclosure provided herein, that combinations of different forms of viruses within one vaccine may, in certain instances, enhance both the humoral and cellular immune responses in the animal that are necessary for protection against a broad spectrum of viral serotypes.

Route and Frequency of Administration:

It has been discovered in the present invention that the route of administration of the vaccine plays a role in the extent of the protective immune response induced by the vaccine in an animal. As will become apparent upon a review of the data presented in the Examples section, a greater level of protective immunity against subsequent challenge by influenza virus was obtained in animals that were administered virus by either a subcutaneous or intradermal route. Thus, while the vaccine of the present invention should not be construed to be limited solely to either of these routes, subcutaneous or intradermal administration of influenza virus is the preferred route and is also a preferred route for non-influenza viruses or other microorganisms or other bioactive agent. However, other routes of administration are also included in the invention, particularly non-natural routes are preferred. By way of non-limiting examples, intramuscular, intratheceal, intraperitoneal, intranasal, rectal, oral, parenteral, topical, pulmonary, buccal, mucosal and other routes of administration are included in the invention for administration of the vaccine of the invention to an animal, particularly if the virus contained within the vaccine is not influenza virus. Routes of administration may be combined, if desired, or adjusted depending on the type of pathogenic virus to which immunity is desired and the site of the body to be protected.

In order to assess the best route of administration of any particular virus when used as a vaccine, the protocols described in the experimental details section may be followed recognizing of course that such protocols are provided as examples only and they should not be construed as having any limiting effect on the invention that is described and claimed herein. These experiments establish that subcutaneous and intradermal vaccination with live virus elicits a very potent immune response directed against influenza virus even at very low doses of virus, without evidence of any clinical signs of disease. These experiments therefore establish that a subcutaneous or intradermal route of administration of a live virus is a useful vaccine strategy against potential pandemics of influenza virus.

Doses or effective amounts of the viral vaccine may depend on factors such as the condition, the selected virus, the age, weight and health of the animal, and may vary among animal hosts. The appropriate titer of virus of the present invention to be administered to an individual is the titer that can induce a protective immune response against the virus, including an antibody and T cell response. An effective titer can be determined using an assay for determining the activity of immunoeffector cells following administration of the vaccine to the individual or by monitoring the effectiveness of the therapy using well known in vivo diagnostic assays.

The vaccine may be administered to an animal as frequently as several times daily, or it may be administered less frequently, such as once a day, once a week, once every two weeks, once a month, or even less frequently, such as once every several months or even once a year or less. Ideally, the vaccine is administered once or at most, twice to the animal. The frequency of the dose will be readily apparent to the skilled artisan and will depend upon any number of factors, such as, but not limited to, the type and severity of the disease being immunized against, the type and age of the animal, etc.

The immunogenicity of a viral vaccine, that is, the generation of antiviral antibody and CD8+ T cell responses in animals that confers on the animal protection from lethal challenge with pathogenic virus strains is determined as described in the experimental examples section herein. Briefly, the vaccine is administered to a group of animals. After a select period of time, the antibody and CD4+ and CD8+ T cell responses are monitored in some animals in the group. Other animals in the group are challenged with pathogenic virus and are monitored for the development of any symptoms of viral disease. The immune response generated and the protective effect conferred by the vaccine to animals subsequently challenged with a pathogenic strain of virus is assessed by comparing the results obtained in animals administered the vaccine as compared with control animals that were not administered the vaccine.

III. Formulations:

Bioactive agents, for example, vaccines produced in accordance with the methodologies described herein can be formulated in a variety of different ways as described herein.

Basic formulations of the bioactive agent include combining the bioactive agent in a pharmaceutical carrier, such as, but not limited to, a chemical composition with which the bioactive agent may be combined and which, following the combination, can be used to administer the bioactive agent to an animal. The pharmaceutical composition may also include any physiologically acceptable ester or salt that is compatible with any other ingredients of the pharmaceutical composition, and which is not deleterious to the animal to which the composition is to be administered. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In these formulations other substances may be included that can be used to form co-polymers, blends or alloys with components of the formulations thus altering the physical properties of the formulation and further modulating the encapsulation/release profiles. These formulations may include substances such as chemokines that attract and retain antigen presenting cells such as dendritic cells or modify the behavior of antigen presenting cells such as Toll-like receptor (TLR) agonists or antagonists.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, gaots, cats, and dogs and other vertebrates, such as birds.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the bioactive agent. The amount of the bioactive agent is generally equal to the dosage of the bioactive agent which would be administered to an animal or a convenient fraction of such a dosage such as, for example, one-half or one-third of such a dosage.

Certain of the formulations included as pharmaceutical compositions of the invention disclosed herein are designed so that the administered bioactive agent is rapidly released into the surrounding tissue, or is slowly released over time. In addition, many of the formulations disclosed herein have the added advantage of retaining the bioactive agent at one temperature, while releasing it at another. For example, a bioactive agent contained in a gel at a temperature below that of body temperature will be retained in the gel, but will be released into the surrounding tissues at body temperature. Further, the bioactive agent of the invention can be formulated to be administered as a single dose in multiple doses. Release profiles and/or single versus multiple dose strategies are determined by those skilled in the art based upon the bioactive agent to be administered. Further the bioactive agent can be released by bursting the material containing them, and/or regulating release by changing the condition by application of energy in the form of ultrasound, light or heat or producing a pH change.

Prior to formulation as described below, bioactive agent, live, attenuated or killed virus may optionally be freeze-dried or lyophilized using lyophilization techniques well known to the skilled virologist and described, for example, in Fields Virology (supra).

The administration of a bioactive agent, such as a live virus vaccine to animals and humans potentially poses a health threat to persons in the immediate vicinity because of the potential for aerosolization of the virus during the injection process. To solve this problem, the present invention includes new delivery formulations and devices that are designed to address the safety concerns of hazardous bioactive agents, such as live virus vaccination. In addition, such formulations and delivery devices provide alternative strategies for release of the bioactive agent into the tissues of the animal. For example, sustained release formulations may be employed, or formulations that release the bioactive agent directly into the tissues may be employed.

Provided herein are encapsulation vehicles comprising non-toxic, natural or synthetic polymers for encapsulation of biologically active agents, such as live virus. Preferably, these polymers are effective for microencapsulation or nanoencapsulation of live virus in combination with microcapsules, nanocapsules or nanotubes. More preferably, these polymers have the added property that when they are combined with bioactive agent, aersolization of the bioactive agent is prevented, thus enhancing the safety of the live virus vaccine while being administered to the animal.

Encapsulation vehicles include, but are not limited to, natural and synthetic polymers such as alginate, hyaluronic acid, xanthan gum, gellan gum, collagen, chitosan, laminin, elastin, Matrigel™, Vitrogen™, polymethyl methacrylate, poly[1-vinyl-2-pyrrolidinone-co-(2-hydroxyethyl methacrylate)], polyvinyl alcohol, poly(vinyl alcohol) (PVA), polyethylene oxide, hydroxyethyl acrylate, polyglyceryl acrylate, acrylic co-polymers (e.g., TRISACRYL); polysaccharides such as dextran and other viscosity enhancing polymers such as carboxy methyl cellulose; polyethylene glycol, polylactic acid and copolymers thereof. Oligomeric compositions of above macromolecules as long as they provide adequate viscoelastic properties to suppress aerosol formation are included.

Further the bioactive agent can itself be contained in any form of microcapsule or nanocapsule known to those skilled in the art, prior to being encapsulated in the vehicle. Such containers include but are not limited to liposomes, polymeric microcapsules for example those composed of poly hydroxy acids, hydrogel capsules or microtubes and nanotubes. Alternatively, bioactive substances contained in a gel can be loaded into microcapsules, nanocapsules or nanotubes.

In one embodiment, the encapsulation vehicle is mixed with the live virus particles and capsules of encapsulated virus small enough to be injected through a needle are generated. Capsule size and the amount of virus in the capsules can be optimized depending on the polymer used, the virus, and the route of administration.

In an alternative embodiment, a cylinder comprising a gelled encapsulation vehicle and live virus is generated inside a single dose needle or other injection device. This process is referred to herein as in-situ gelation. The in-situ gelation approach provides a ready-to-use unit capable of injecting a very small cylinder of encapsulated virus subcutaneously or intradermally. In this embodiment, the encapsulation vehicle is designed to achieve a desired gel strength based upon the selected route of delivery of the vaccine so that it remains a solid injectable gel at room temperature but releases the encapsulated virus at body temperature, or in a pre-programmed release profile. The needle size, initial gel concentration, means for dislodging the cylinder, and amount of virus contained therein is optimized depending on the type of polymer used, the virus, and the route of administration.

Temperature and pH are other possible factors that can be varied in order to achieve the desired properties of aerosol suppression, appropriate viscoelastic properties and release kinetics. Furthermore, an encapsulation vehicle comprised of gel and microcapsule, nanocapsule or nanotube can be subjected to control release of the bioactive agent by bursting the containers and/or regulating release by changing the condition by application of energy in the form of ultrasound, light or heat or producing a pH change.

Exemplary polymers for use in encapsulating the live virus are described in more detail below, and include, without limitation, alginates, hyaluronic acid, cellulose, dextrans and collagen matrices.

Methodologies that are designed to generate encapsulating polymers for other applications can be adapted to the present invention, provided that when the virus to be delivered is a live virus, the virus must not become substantially inactivated and/or lose immunogenicity while in the encapsulated state. Similar restrictions apply to bioactive agents where encapsulation must preserve the desired activity.

By the term “substantially inactivated” is meant that the virus must retain at least some infectivity and therefore be capable of infecting and replicating in a host cell.

It will be understood by the skilled artisan that the encapsulation vehicles described herein are useful not only for live virus vaccination strategies, but are also useful for the safe delivery of any biologically active agent to a subject.

Non-limiting examples of encapsulation vehicles are now described. The experimental conditions useful to generate encapsulation vehicles and their use in a vaccine is described more fully in the experimental examples section elsewhere herein. The encapsulation vehicle useful in the present invention confers a level of safety on the bioactive agent by preventing aerosolization of the virus during administration. Further, the encapsulation vehicle facilitates the generation of immediate or sustained release formulations of the bioactive agent. In addition, virus may be safely stored in the encapsulation vehicle prior to use, whether or not the bioactive agent/encapsulation vehicle combination is preloaded in a delivery device prior to administration.

The invention includes the use of a gelatin polymer as an encapsulation vehicle for the viral vaccine of the invention. The concentrations of gelatin that are useful in the vaccine of the invention may range from about 0.05 to about 25% (w/w) of gelatin to water and any and all whole or partial integers therebetween. Various concentrations of gelatin are mixed with virus and the resulting solution is loaded into a delivery device, for example, but not limited to a needle or a syringe. Gelling of the gelatin prior to during, or after loading, is induced following procedures known in the art. The advantage to the use of gelatin in the present invention is the fact that crosslinking of the gelatin can occur in the absence of any added chemical agent in that crosslinking occurs via temperature alone as the crosslinking agent.

The virus gelatin mixture is inoculated into animals and the effect on the immune response is assessed as described more fully elsewhere herein. Preferably, the gelatin is lyophilized and irradiated with γ rays or other sterilization method, in order to sterilize it. The concentration of gelatin to be used will vary depending on the desired release rate of virus from the gel following administration to the animal and will be apparent to the skilled artisan once armed with the present invention.

Optionally, a co-polymer, polymer blend or alloy, such as, but not limited to polyethylene glycol (PEG) may be used in conjunction with the gelatin. PEG acts to reduce water loss from the gel. PEG sizes ranging from about 500 to about 50,000 are useful in the invention and any and all whole or partial integers therebetween, with preferred molecular weights being about 100, 2,000, 3,000, 4,000, 5,000, 6,000, 7,000, 8,000, 9,000 and 10,000 of PEG. PEG can be included in the gelatin virus mixture at approximately 0.1-20% w/w of PEG/gelatin, although this range may vary depending on any number of factors, including but not limited to the strength of the gelatin, the virus used in the vaccine, the route of delivery, and the like. Thus the range of 0.1-20% w/w of PEG/gelatin should be construed to include any whole or partial integers therebetween.

An encapsulation vehicle comprising a collagen gel may also be used in the present invention. A collagen gel may be synthesized and characterized as described elsewhere herein. The concentration of collagen useful in a gel for vaccine production may vary from about 0.5 to about 50 mg/ml of collagen solution and any and all whole or partial integers therebetween. Crosslinking of collagen is accomplished using techniques well known to the skilled artisan and is more fully described elsewhere herein.

The virus collagen mixture is inoculated into animals and the effect on the immune response is assessed as described more fully elsewhere herein. Preferably, the collagen is lyophilized and can be irradiated with y rays in order to sterilize it. The concentration of collagen to be used will vary depending on the desired release rate of virus from the gel following administration to the animal and will be apparent to the skilled artisan once armed with the present invention.

Optionally, a co-polymer, polymer blend or alloy, such as, but not limited to polyethylene glycol (PEG) may be used in conjunction with the collagen. PEG sizes ranging from about 500 to about 50,000 are useful in the invention and any and all whole or partial integers therebetween, with preferred molecular weights being about 100, 2,000, 3,000, 4,000, 5,000, 6,000, 7,000, 8,000, 9,000 and 10,000 of PEG. PEG can be included in the collagen virus mixture at approximately 0.1-20% w/w of PEG/collagen, although this range may vary depending on any number of factors, including but not limited to the strength of the collagen, the virus used in the vaccine, the route of delivery, and the like. Thus the range of 0.1-20% w/w of PEG/collagen should be construed to include any whole or partial integers therebetween.

The encapsulation vehicle of the present invention may also include alginate. Alginates of varying viscosities are available by virtue of their molecular weight. It is also possible to produce solutions of different viscosities by varying the concentration and type of alginate. Cross-linked gels of various strengths can be produced by varying the concentration and type of the ion crosslinker as described in the experimental sections herein. Differing types of alginates also include those that have varying compositions of the guluronic:manuronic acid ratio in the polymer backbone. Methods for assessing the optimal alginate composition for use in the present invention are presented in the experimental examples section herein.

Typically, alginate concentrations range from about 0.1% to about 20% of alginate and any whole or partial integer therebetween. Preferred concentrations include about 0.5%, 1%, or 1.5 or 2% or up to 20% alginate depending on the type of alginate used. Alginate can be combined with other polymers such as chitosan to produce gels having desired properties. It can also interact with polycations such as poly-L-Lysine. Modified alginates are also available such as PEG-alginate.

As discussed elsewhere herein, a preferable vaccine is one that the vehicle comprises of gel, solution or powder loaded into microcapsules, nanocapsules or nanotubes. The invention therefore includes the synthesis and loading of microcapsules with dimensions in the order of one to several hundred microns, with preferred size of 10-100 microns. The invention also includes the synthesis and loading of nanocapsules with one dimension being less than 1 nm up to 1 microns, with preferred size of 100 nm-1 micron. The invention also includes the synthesis and loading of nanotubes, wherein the nanotubes have a range of diameters from about 1 nm to 1000 nm, preferably 20 to 500 nm and any and all whole or partial integers therebetween. Nanotubes having diameters larger than 200 nm facilitate the generation of structures that can retain and release particles the size of an influenza virus. A preferred nanotube for use in the present invention is a multi-wall nanotube (MWNT). Nanotubes can be synthesized using technology available to the skilled artisan and disclosed for example in Miller et al. (Miller et al., 2001, J. Amer. Chem. Soc. 123:12335-12342).

Virus that is loaded into the nanotubes of the present invention should not appreciably diffuse out of the lumen of the tube. Preferably, the nanotubes are loaded with fluids that are more viscous than water. Further, the nanotubes may be loaded with virus that is encapsulated in a gel or contained in a viscous or semi-viscous solution and described elsewhere herein, for example, in a gelatin, collagen, alginate or other gel that is capable of releasing virus into the surrounding tissue at body temperature, thereby conferring added safety and other advantages to the vaccine of the invention. Nanotubes can be loaded with a powder, for example, but not limited to a freeze-dried bioactive agent. The nanotubes loaded internally with bioactive agent encapsulated gels can be administered safely using any of the devices described in this invention or using any other devices capable of producing the desired effect of preserving safety of delivery. Alternatively, nanotubes loaded with the bioactive agent can be encapsulated into a gel and then delivered using various devices as described more fully below.

IV. Devices:

The invention includes the use of various devices for storage and delivery of the bioactive agent to an animal, preferably a mammal and more preferably, a human. Such devices include, without limitation, needles, syringes, catheters, gene guns, nanotubes, patches, mucosal applicators, and the like, that are preloaded with the desired bioactive agent formulation, i.e., prior to administration of the same to the animal, or are loaded with the bioactive agent vaccine at the time of administration of the bioactive agent to the animal.

The invention includes the use of any and all devices that are presently known, or yet to be discovered, that perform the function of penetrating the tissue of an animal and delivering an agent into the internal animal tissue. Thus, the invention includes any and all single or plurality of needles, syringes, needle syringe combinations, gene guns, and the like.

Each of these devices can be loaded with bioactive agent alone or bioactive agent that is encapsulated as described herein. Loading of these devices can occur immediately prior to administration of agent to the animal, or can be conducted elsewhere and the devices are then stored until shipping and use.

As discussed elsewhere herein, a preferable vaccine is one that is loaded into nanotubes. The invention therefore includes the synthesis and loading of carbon nanotubes, wherein the nanotube have a range of diameters from about 50 nm to 250 and any and all whole or partial integers therebetween. Larger diameter nanotubes facilitate the generation of structures that can retain and release particles the size of an influenza virus. A preferred nanotube for use in the present invention is a multi-wall nanotube (MWNT). Nanotubes can be synthesized using technology available to the skilled artisan and disclosed for example in Miller et al. (2001, J. Amer. Chem. Soc. 123:12335-12342). Virus that is loaded into the nanotubes of the present invention should not appreciably diffuse out of the lumen of the tube. Preferably, the nanotubes are loaded with fluids that are more viscous than water. Further, the nanotubes may be loaded with virus that is encapsulated in a hydrogel and described elsewhere herein, for example, in a gelatin, collagen, alginate or other hydrogel that is capable of releasing virus into the surrounding tissue at body temperature, thereby conferring added safety and other advantages to the vaccine of the invention.

V. Methods:

The invention additionally includes a method of eliciting a CD8+ T cell and/or antibody immune response in a vertebrate, preferably a human. The method comprises administering to the vertebrate a vaccine comprising a CD8+ T cell and/or antibody immunoprotective amount of virus, whereby a CD8+ T cell and/or antibody immune response is elicited in the vertebrate. The virus to be administered to the vertebrate is a respiratory virus and is preferably an influenza virus type A. The route of administration is any route, and when the virus is influenza virus type A, the preferred route of administration is subcutaneously or intradermally. The virus may be administered in a pharmaceutically acceptable composition as that term is defined herein, or in any of the encapsulation formulations and using any of the devices described elsewhere herein. To determine whether a CD8+ T cell and/or antibody response has been elicited in the vertebrate, the procedures disclosed in the experimental examples herein are followed.

Also included in the invention is a method of protecting a vertebrate against infection by a virus. This method comprises administering to the vertebrate a vaccine comprising a CD8+ T cell and/or antibody immunoprotective amount of the virus, whereby a CD8+ T cell and/or antibody immune response is elicited in the vertebrate thereby protecting the vertebrate against the infection. The virus to be administered to the vertebrate is a respiratory virus and is preferably an influenza virus type A. The route of administration is any route, and when the virus is influenza virus type A, the preferred route of administration is subcutaneously or intradermally. The virus may be administered in a pharmaceutically acceptable composition as that term is defined herein, or in any of the encapsulation formulations and using any of the devices described elsewhere herein. Protection of a vertebrate against subsequent virus infection is described elsewhere herein.

Further included is a method of preventing a virus infection in a vertebrate. The method comprises administering to the vertebrate a vaccine comprising a CD8+ T cell and/or antibody immunoprotective amount of virus, whereby a CD8+ T cell and/or antibody immune response is elicited in the vertebrate thereby preventing a virus infection in the vertebrate. The virus to be administered to the vertebrate is a respiratory virus and is preferably an influenza virus type A. The route of administration is any route, and when the virus is influenza virus type A, the preferred route of administration is subcutaneously or intradermally. The virus may be administered in a pharmaceutically acceptable composition as that term is defined herein, or in any of the encapsulation formulations and using any of the devices described elsewhere herein.

In addition, there is included a method of treating a virus infection in a vertebrate. The method comprises administering to the vertebrate a vaccine comprising a CD8+ T cell and/or antibody immunoprotective amount of virus, whereby a CD8+ T cell and/or antibody immune response is elicited in the vertebrate thereby treating the vertebrate against the infection. The virus to be administered to the vertebrate is a respiratory virus and is preferably an influenza virus type A. The route of administration is any route, and when the virus is influenza virus type A, the preferred route of administration is subcutaneously or intradermally. The virus may be administered in a pharmaceutically acceptable composition as that term is defined herein, or in any of the encapsulation formulations and using any of the devices described elsewhere herein. This method of the invention is particularly useful in the event of a pandemic, especially in humans. Vaccine can be administered to a human at the onset of symptoms in order to treat the human and prevent more serious illness.

The invention also includes a method of enhancing safety when administering a bioactive agent to an animal. The method comprises administering a composition comprising a CD8+ T cell immunoprotective and/or antibody immunoprotective amount of a bioactive agent to the animal, wherein the bioactive agent induces an immunoprotective CD8+ T cell and/or antibody response in the animal following administration of the bioactive agent by a route that does not cause disease in the animal and further wherein the bioactive agent is encapsulated in an encapsulation vehicle.

Also included is a method of enhancing safety when administering a bioactive agent to an animal where the method comprises administering to the animal a composition comprising a CD8+ T cell immunoprotective amount of bioactive agent. The bioactive agent induces an immunoprotective CD8+ T cell response in the animal following administration of the bioactive agent by a route that does not cause disease in the animal.

In each of these methods, the bioactive agent is encapsulated in an encapsulation vehicle and the bioactive agent is selected from the group consisting of a microorganism and a protein.

Also included is a method of enhancing safety when administering a bioactive agent to an animal. The method comprises administering to an animal a composition comprising an amount of a bioactive agent that induces a desired response while reducing risk in an animal. The route of administration of the bioactive agent is a route that does not cause disease in the animal, and further the bioactive agent is encapsulated in an encapsulation vehicle thereby enhancing safety when administering the bioactive agent.

Each of the methods of the invention can be conducted on any animal, preferably a human, including the very old, the very young and any otherwise immunocompromised human, and well as healthy humans.

VI. Other Compositions:

The invention further includes a composition comprising a biologically effective amount of a bioactive agent, wherein the bioactive agent induces a desired response in an animal while reducing risk in an animal following administration of the bioactive agent to the animal by a route that does not cause disease in the animal. Preferably, the route is a non-natural route and more preferably, the the route is selected from the group consisting of subcutaneous, intradermal, intramuscular, mucosal and oral.

The bioactive agent in the composition may be encapsulated in an encapsulation vehicle and may also be associated with a nanotube, a liposome or a protein prior to being encapsulated in the encapsulation vehicle. The encapsulation vehicle is at least one member selected from the group consisting of a gel, a liquid or a powder and may also be loaded into a microcapsule, nanocapsule or nanotube. The encapsulation vehicle may comprise a polymer and preferably, the polymer is not toxic when administered to an animal. The polymer is preferably associated with the bioactive agent thereby delaying release of the bioactive agent into the surrounding environment. More preferably, the the polymer is a gel. The bioactive agent is preferably selected from the group consisting of a microorganism, and a protein.

VII Kits:

The invention includes various kits which comprise the bioactive agents and vaccines of the invention. Also included in the kit of the invention are instructional materials which describe use of the vaccine in the methods of the invention. Although exemplary kits are described below, the contents of other useful kits will be apparent to the skilled artisan in light of the present disclosure. Each of these kits is included within the invention.

The invention includes kits for use in a method of: eliciting a CD8+ T cell and/or antibody immune response in a vertebrate; for protecting a vertebrate against infection by a virus; for preventing a virus infection in a vertebrate; and for treating a virus infection in a vertebrate.

The kit of the invention comprises a device that may be preloaded with bioactive agent ready to be administered to an animal and instructional materials for the use thereof. Alternatively, the kit includes a device, a preparation of bioactive agent that may or may not be freeze-dried, a solution for suspension of the bioactive agent and instructional material for the combination of the device, bioactive agent and solution, and further instructions regarding the administration of the same to a vertebrate, preferably a human. The bioactive agent may be suspended in a pharmaceutically acceptable carrier, optionally including an encapsulation formulation. The pre-loaded device may be a needle, and syringe, a needle and syringe combination, or a nanotube, or any combination of the foregoing.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The experimental methods useful in the invention are first described below.

Animals, Antibodies and Influenza Virus Infections

Animal studies were conducted under IACUC approval. Specific pathogen-free 8-12 week old C57BL/6J (B6) wild type mice are available from Jackson Laboratories (Cincinnati, Ohio). C57Bl/10SgSnAiRag^{-/-}γ^{-/-} (henceforth denoted Rag−/−γc−/−) and C57Bl/10 female mice are available from Taconic (Germantown, N.Y.). All mice were maintained in AAALAC certified barrier facilities at Drexel University College of Medicine, Drexel University. As a general rule, nine animals were included in each group. Influenza A/Puerto Rico/8/34 (PR8) viral strain (H1N1) and the X31 recombinant strain of A/Aichi/2/68 and A/Puerto Rico/8/34 (H3N2) were used in the experiments. These virus strains express different surface hemagglutinin (H) and neuraminidase (N) proteins and therefore antibody response against these viruses do not cross-react with each other which is important in experiments in which the animals were rechallenged. The six internal genes of these viruses are similar between the different strains, thus allowing for the assessment CTL responses in rechallenge experiments. Generally, mice were infected intranasally with twelve hemagglutination units (HAU) of virus strain X31. In the case of a secondary response, mice were injected IP with PR8 strain (1000 HAU) and then re-challenged intranasally with 12 HAU of the X31 strain. Safety and rechallenge studies were performed using the A/Equine/London/1416/73 virus (H7N7) that is highly virulent in mice. Preparation of lung and spleen mononuclear cells, phenotypic analysis of NP₃₆₆-specific CD8+ T cell, intracytoplasmic cytokine staining, flow cytometry, cytotoxicity assays, anti-influenza antibody titers and pulmonary viral titer assay were performed as previously described (Halstead et al., 2002, Nat. Immunol. 3:536-541).

Preparation of Lung, Spleen Mononuclear Cells

Lungs were digested at 37° C. for 90 minutes in 1 mg/ml Collagenase A (Roche Molecular Biochemicals, Indianapolis, Ind.) and 40 U/ml DNAse (Sigma, St Louis, Mo.) and the resulting tissue was passed through a 100 μm nylon mesh and then was washed. Spleens were homogenized into single cell suspensions using Corningware Glass tissue disruptors (Fisher Scientific, Pittsburgh, Pa.). Lymphocytes obtained from both lung and spleen suspensions were then separated by density gradient centrifugation using Hystopaque 1083 (Sigma, St Louis, Mo.).

Phenotypic Analysis and Measurement of NP366-Specific CD8+ T Cell

Cells were stained with APC-labeled NP₃₆₆ tetramers. Cells were also co-stained with combinations of FITC-, Cy5PE- and PE-conjugated antibodies to surface markers i.e. anti-CD3-PE (Becton-Dickinson, San Jose, Calif.), anti-CD8-FITC (eBioscience, San Diego, Calif.) and anti-CD4-CyChrome (Cy5PE) (eBioscience) for 30 minutes on ice. Following washes and fixation in paraformaldehyde, 2×10⁵ cells were analyzed by flow cytometry using a FACS Calibur® (BD Biosciences) and FlowJo software (Treestar, San Carlos, Calif.). In some cases, up to 10 color analysis of NP₃₆₆-specific CD8+ T cells can be performed using a 3-laser FACSAria® high-speed cell sorter (BD Biosciences).

Biotinylated H-2D^{b}/β2 m/Peptide Complexes

Biotinylated H-2D^{b}/β2 m/peptide complexes were produced as described (Altman et al., 1996, Science 274:94-96). The H-2D^{b}-restricted Influenza type A nuclear protein NP(366-374) immunodominant epitope (ASNENMETM (SEQ ID NO:1)) (Townsend et al., 1986, Cell 44:959-968) was complexed into the H-2D^{b} to produce the NP366 tetramers used in these studies.

Cytotoxicity Assays

EL-4 cells (ATCC) were loaded with peptides by incubating for 6 hrs at 37° C. with 1 μg/ml of the Influenza type A peptides NPP₃₆₆₋₃₇₄, NS2₁₁₄₋₁₂₁, M1₁₂₈₋₁₃₅ and PA₂₂₄₋₂₃₃. Following incubation, cells were washed and labeled with Na₂⁵¹CrO₄ (NEN, Boston, Mass.) for 75 minutes at 37° C. and then washed again. These EL4 cells were then added at 10⁴ (100 μl)/well to a 96-well round-bottom microtiter plate (Falcon, Becton-Dickinson Labware, Franklin Lakes, N.J.). Effector and target cells were then plated at ratios of 100:1, 50:1, 25:1 and 10:1, and incubated for 6 hrs at 37° C. Plates were subsequently centrifuged and 30 μl of supernatants were transferred to 96-well LumaPlates (Packard, Meriden, Conn.) and counted in a TopCount microplate scintillation counter (Packard). Specific cytotoxicity was determined using the formula:
% Specific Lysis=(Experimental CPM−Spontaneous CPM)×100(Maximum CPM−Spontaneous CPM)

Maximum ⁵¹Cr release was determined by lysing target cells with 5% Triton X-100 (Sigma, St Louis, Mo.). Spontaneous ⁵¹Cr release was determined using target cells incubated with media alone.

Intracytoplasmic Cytokine Staining

For intracytoplasmic cytokine staining, 10⁶/ml/well lung lymphocytes, spleen mononuclear cells, or purified CD8+ T cells were stimulated with 10 μg/ml NPP₃₆₆₋₃₇₄, NS2₁₁₄₋₁₂₁, M1₁₂₈₋₁₃₅ and PA₂₂₄₋₂₃₃ peptide, anti-CD3 antibody or PMA (25 ng/ml)+lonomycin (1 μg/ml) in the presence of 2.5 μM Monensin for 5 hours and then fixed with 4% paraformaldehyde for 10 minutes at 4° C. Cells were washed twice and permeabilized with 0.1% Saponin at 4° C. for 10 minutes. Cells were then washed and incubated with anti-IFNγ antibody (eBioscience) at 4° C. for 30 minutes. Cells were washed and fixed in 1% paraformaldehyde and then 2×10⁵ events were collected on a FACS Calibur® (BD Biosciences) and analyzed with FlowJo software.

Influenza Viral Titer Assay

Lungs were homogenized and viral supernatants were collected following centrifugation of the homogenate at 1500×g for 15 minutes and frozen at −80° C. until subsequent analysis. Dilutions of viral supernatants were added to 3×10⁴ Madin Darby canine kidney (MDCK) cells/well a 96-well U-bottom plate. After infection of the MDCKs for 24 hours at 37° C., media was aspirated from each well (MDCKs are adherent cells) and serum-free media was added. Virus titers were determined four days later by determining the dilution at which the supernatants no longer agglutinate chicken red blood cells using standard curve of known virus concentration and the Reed-Munch calculation of TCID. A second method that can be used to measure viral titers utilizes Real Time PCR as previously described (Ward et al., 2004, J. Clin. Virol. 29:179-188).

Identification of a Robust Immune Response

Specific criteria that are used to identify a robust immune response upon administration of vaccine to mice are: Upon intranasal rechallenge with influenza virus, there should be observed the induction of >20% NP₃₆₆-specific CD8+ T cells of total CD8+ T cells or >1×10⁶ NP₃₆₆-specific CD8+ T cell in the lungs on day seven of rechallenge and/or >1/500 titer of serum neutralizing antibodies (in a hemagglutination inhibition assay). A non-previously immunized naïve mouse generally exhibits <3% NP₃₆₆-specific CD8+ T cells and <10⁵ NP₃₆₆-specific CD8+ T cells in the lung on day seven of infection.

Statistical Analysis

Data are analyzed using the Mann-Whitney U test, Wilcoxon's signed-rank test for paired data, Student's t test and Spearman's rho correlation using JMP Statistics Guide (SAS Institute Inc., Cary, N.C.).

Safety

The safety of any vaccine can be tested in wild type and RAG−/−γc−/− animals. Safety is evaluated by examining the general appearance of the animals, weight loss, and by assessing the pathology of lung and other tissues. Viral loads obtained from lungs, spleen, liver and brain are assessed using Real time PCR. These studies can be conducted using PR8 and A/Equine/London/1416/73H7N7 virus (London strain) following SQ or ID delivery of live virus. Nine animals can be included in each group. The use of the highly pathogenic A/Equine/London/1416/73 virus which causes systemic and brain infection when administered intranasally (Kawaoka, 1991, J. Virol. 65:3891-3894; Christensen et al., 2000, J. Virol. 74: 11690-11696) provides a very stringent test for assessment of viruses administered SQ and ID. Animals may be observed and weighed daily for 30 days. Animals are immunized with 1, 0.1, 0.01 and 0.001 HAU of virus ID or SQ and followed for 30 days. Animals are evaluated for clinical signs and weight loss. Animals are monitored twice daily by visual inspection. Animals are weighed daily. Animals are removed when they meet the following criteria: 1) unresponsive to extraneous stimulation, 2) prostration for >1 hour, 3) labored breathing, 4) persistent tremors, 5) animals persistently hunched. All observations are recorded. Animals removed will be counted non-survived in survival analysis. Death is not an endpoint for these studies. Animals are followed for 30 days. A vaccine is considered safe when it does not induce more than 5% weight loss in animals which is within the experimental error of such measurements and results in 90% survival of the animals.

Virus Infection and Route of Administration

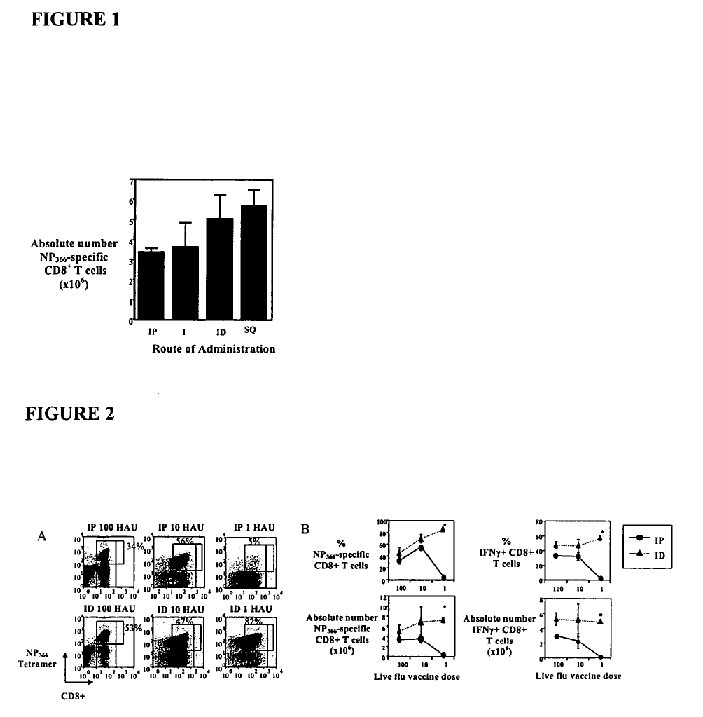

To study live virus vaccination, the effect the route of administration may have on the antiviral CD8+ T cell response was investigated. Eight week old mice were immunized intraperitoneally (IP), intramuscularly (IM), intradermally (ID), or subcutaneously (SQ) with the PR8 strain of influenza type A virus and were rechallenged intranasally 3045 days later with the X31 strain of influenza virus. Mice were sacrificed at the peak of the secondary response (day 7) and the influenza virus nuclear protein (NP₃₆₆) virus-specific CD8+ T cell response was examined. FIG. 1 depicts that administration of virus by the ID and SQ routes resulted in a stronger virus-specific CD8+ T cell response in the lungs than did administration of virus by the IM or IP routes. The numbers shown in the FIG. 1 are the mean±SE and are 5.04±1.17×10⁶ and 5.71±0.79×10⁶ virus-specific CD8+ T cells for ID and SQ routes, respectively, compared with 3.65±1.21×10⁶ and 3.41±0.18×10⁶ virus-specific CD8+ T cells, respectively, for the IP and IM routes. These results indicate that the route of immunization with live virus affects the extent of the overall antiviral CD8+ T cell response, and that the ID route induces the most potent response. Most importantly, it was observed that the use of an ID or SQ route of administration of live influenza virus to a mouse did not result in clinical disease in the animals. Next, a dose response study was performed to determine if the observed differences in the immune response were evident at much lower doses of virus. Mice were administered live influenza virus by IP or ID injection using decreasing concentrations of virus. The virus-specific response to secondary rechallenge by influenza virus was then examined. Representative FACS plots depicting pulmonary virus-specific CD8+ T cells (NPP+CD8+) in the lungs of IP and ID primed mice are shown in FIG. 2. Decreasing doses of live influenza virus administered to mice IP, resulted in a decreased number of virus-specific CD8+ T cells in the lung as measured by MHC class I tetramer (3.65±1.21×10⁶ cells at the high dose and only 0.35±0.1×10⁶ cells at the lowest dose) and NP₃₆₆ peptide specific IFNγ producing CD8+ T cells (2.81±0.1×10⁶ at the high dose, and 0.18±0.05×10⁶ at low dose.) Decreasing doses of live influenza virus administered ID resulted in an increase in the antiviral CD8+ T cell response (5.05±1.18×10⁶, high dose, vs. 7.2±0.46×10⁶, low dose, virus-specific CD8+ cells and 4.06±0.79×10⁶, high dose, vs. 4.83±0.24×10⁶, low dose, IFNγ producing CD8+ T cells.) Thus, administration of a low dose of live influenza virus IP, induced a weak virus specific CD8+ T cell response, while administration of the same dose ID elicited a very strong virus specific CD8+ T cell response. These results demonstrate the increased efficiency of ID administration as compared with IP administration of live influenza virus in inducing a virus-specific CD8+ T cell response and they indicate that very low doses of live virus can elicit very potent responses when the ID or SQ routes are used.

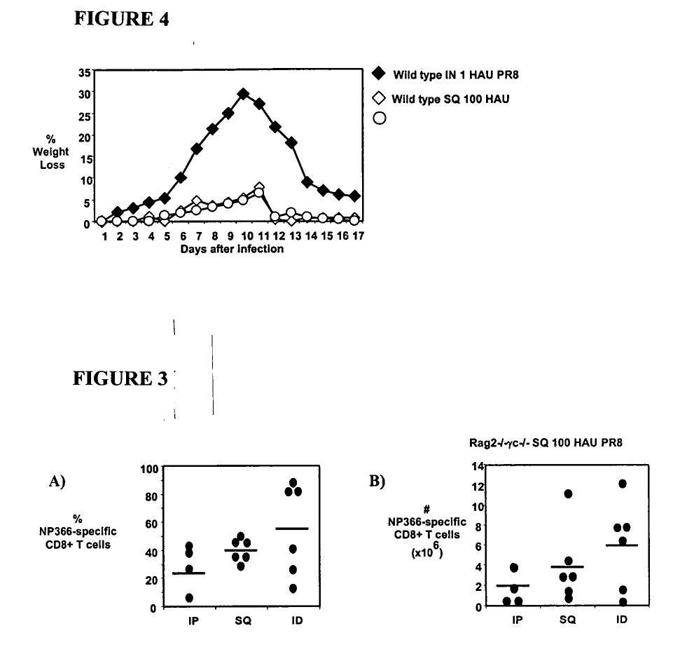

To further compare ID and SQ immunizations, eight week old C57Bl/6J mice were immunized intraperitoneally (IP), subcutaneously (SQ), or intradermally (ID) with a low dose (1 HAU) of live influenza virus type A strain PR8 (H1N1) and rechallenged intranasally forty five days later with influenza virus heterosubtype X31 (H3N2). Following rechallenge, mice were sacrificed at the peak of the secondary immune response (day seven after rechallenge). The NP₃₆₆-specific CD8+ T cell response was assessed using MHC class I tetramers loaded with a peptide (NP366) spanning amino acids 366-374 (ASNENMETM (SEQ ID NO: 1)), corresponding to the immunodominant epitope derived from NP (Flynn et al., 1998, Immunity 8:683-691). The ID and SQ routes of administration resulted in a stronger immune response in the lungs of the rechallenged mice when compared with mice that were immunized IP, based on the percentages and total numbers of NP-specific CD8+ T cells recovered (FIG. 3A). NP₃₆₆-specific CD8+ T cells were recovered from the lungs of mice that were immunized SQ (n+6) or ID (n+6) at concentrations of 3.76±3.7×10⁶ and 5.8±4.3×10⁶ cells, respectively. In contrast, only 1.9±1.6×10⁶ NP-specific CD8+ T cells were recovered from IP immunized mice (n=5) (FIG. 3B). These results indicate that low doses of live virus elicit a strong CD8 T cell response, especially when delivered ID or SQ. Bearing in mind that SQ immunization in humans is easy to perform, it was decided that this route of delivery would be used for initial testing of the vaccine for efficacy and safety.

To test whether SQ administration of live virus induces disease in host animals, wild type C57Bl/6J mice and mice that lack T, B and NK cells (strain C57Bl/10SgSnAiRag^{-/-}γc^{-/-}, henceforth denoted Rag−/−γc−/−) were immunized. The latter mice were unable to mount an NK-mediated or an adaptive immune response to the virus. Following subcutaneous administration of 1 HAU live PR8 virus, over a period of thirty days, neither the Rag−/−γc−/− nor the C57Bl/6J mice exhibited any signs of infection and in addition, neither set of mice lost weight which is indicative of an active viral infection. To test whether a dose of 100 HAU live PR8 virus could induce disease in Rag−/−γc−/− or C57Bl/6J mice, five Rag−/−γc−/− mice and 5 C57Bl/6J mice were immunized with 100 HAU PR8 influenza virus and their weight was recorded for seventeen consecutive days. SQ injection of a viral dose that was one hundred times higher than that proposed for routine immunization was also safe and did not induce weight loss in C57Bl/6J or Rag−/−γc−/− mice (FIG. 4). However, intranasal (IN) infection of C57Bl/6J mice with 1 HAU PR8 influenza virus induced infection and progressive weight loss in C57Bl/6J mice (n=5) beginning at day six and peaking at day ten after infection, as expected. Beginning on day eleven after intranasal infection, the C57BL/6J mice began to recover (FIG. 4).

The data thus far indicate that the SQ route for administration of live virus to a mammal does not cause active viral infection or systemic disease in wild type or immunodeficient mice. This route of administration is therefore considered to be safe. Without wishing to be bound by theory, these findings support studies that suggest that the control point during systemic infection with influenza virus is the level of viremia that follows respiratory infection (Lu et al., J. Virol. 73:5903-5911). These data also support the hypothesis that the administration of live virus via an alternative, non-natural route does not result in overt disease unless administration is by a direct intravenous route (Swayne and Slemons, 1994, Vet. Pathol., 31:237-245).

Additional Safety Data

Additional data that support the feasibility and efficacy of the live virus vaccine strategy are now presented. The live influenza virus strategy has been tested using highly pathogenic strains of influenza virus (London strain). The data obtained establish that subcutaneous delivery of the virus to a mammal is safe. In addition, the data establish that live influenza virus entrapped in alginate gel elicits a potent influenza-specific CD8+ T cell response in the recipient mammal. Importantly, the data further establish that administration of live virus subcutaneously to a mammal elicits an antibody response wherein neutralizing antibody titers are induced in the mammal following administration of a single dose of virus. In addition, the data presented herein establish that it is possible to load nanotubes with alginate gels that contain 50 nm size Quantum dots (QDots), thus demonstrating that loading virus plus alginate into nanotubes is feasible. Further, it is established herein that sonnication under the conditions described elsewhere herein, breaks nanotubes into smaller, more preferred fragments.

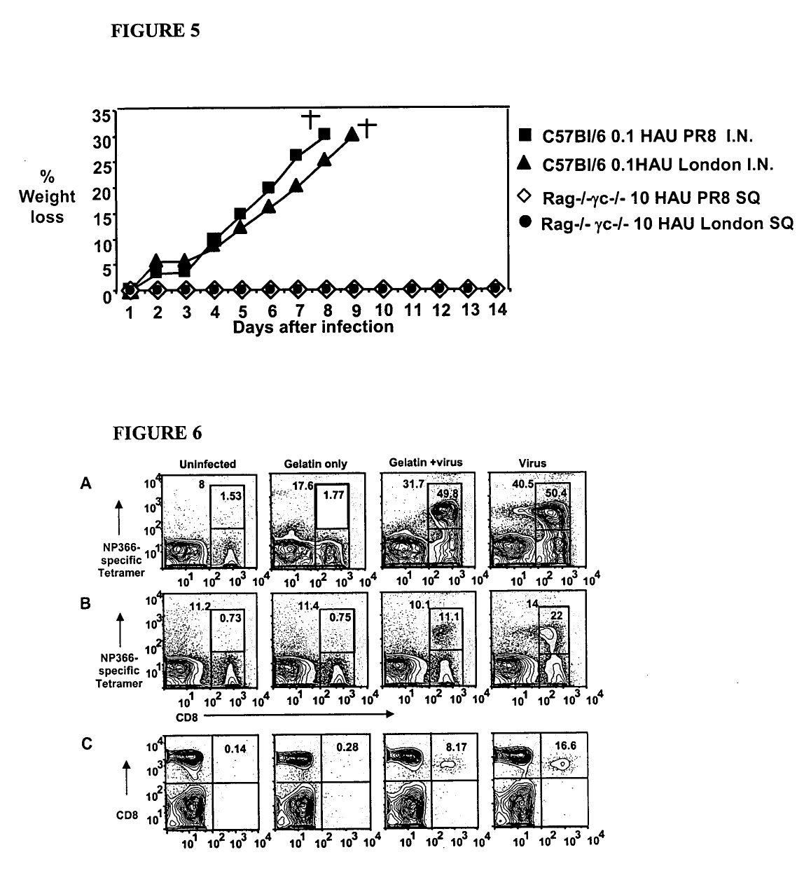

Additional data on the safety of delivering live influenza virus subcutaneously into RAG−/−γc−/− (mice that have no T cells, B cells or NK cells) and wild type mice are described below. In FIG. 5, wild type and RAG−/−γc−/− animals (n=5 in all groups) were infected with a new and more potent batch of PR8 influenza virus or the highly pathogenic A/Equine/London/1416/73H7N7 virus (London strain). Wild type animals that were infected intranasally with 0.1 HAU of PR8 or the London strain exhibited symptoms of influenza virus infection and lost up to 30% of their body weight. At this time, certain animals were euthanized as they had become moribund. In contrast, when RAG−/−γc−/− mice (FIG. 5) and wild type animals were inoculated subcutaneously with 10 HAU live virus (that is, 100 times the dose used in the intranasal infections), they did not lose any weight and exhibited no signs of illness for more than the thirty days for which they were followed. These data establish that the route of administration of the virus is critical for the safety of the inoculation procedure.

Efficacy and Safety of Vaccine Delivered in Gelatin Polymer Gel

To test the basic premise that live virus in a polymer gel can retain its immunogenicity, a 3% (w/w) solution of gelatin was mixed at 30° C. with live PR8 influenza (10 HAU/μl) virus in PBS and was loaded into a 1 ml syringe. The gelatin/virus ratio was established so that each 100 μl gelatin gel contained about 10 HAU live virus. The gelling of the gelatin was induced by incubating the syringe on ice for 30 minutes. C57Bl/6 mice were immunized subcutaneously with 100 μl gelatin mixed with live virus (n=2) or 100 μl gelatin (n=2) only. As a control, a group of C57Bl/6J mice (n=2) were immunized subcutaneously with 10 HAU of PR8 live virus. Thirty days after immunization, the mice that had received live virus in gelatin were rechallenged with live influenza virus strain X31 administered intranasally. At the peak of the secondary immune response (seven days after rechallenge), the frequency and total number of NP-specific CD8+ T cells isolated from the lungs (FIG. 6A) and spleens (FIG. 6B) of the rechallenged mice was assessed. In the lungs of mice that had been immunized subcutaneously with 10 HAU live virus contained in gelatin gel (3% w/w) or with 10 HAU live virus alone, there was a massive accumulation of CD8+ T cells (percentage written outside CD8+ gate, FIG. 6A) that represented about 30-40% of the total number of lymphocytes present. Moreover, about half of the CD8+ T cells isolated from the lungs of mice that received virus only or virus incorporated in gelatin gel were specific for the immunodominant NP366 viral epitope (percentage written inside NP₃₆₆-specific CD8+ T cell gate, FIG. 6A). In contrast, in mice that received gelatin only, the number of CD8+ T cells infiltrating the lungs represented only about 17% of the total lymphocytes present and the percentage of NP₃₆₆-specific CD8+ T cells present was only 2% which is consistent with a day seven primary immune response to the virus (FIG. 6A). When the spleens of mice immunized with live virus contained in gelatin were examined, about 10% of the CD8+ T cells were NP₃₆₆-specific CD8+ T cells (FIG. 6B). In contrast, in mice that were immunized with virus only, about 22% of the total CD8+ T cells were NP₃₆₆-specific. The percentage of NP₃₆₆-specific CD8+ T cells in the spleens of mice immunized with gelatin was below 1% (FIG. 6B).

The next set of experiments were conducted to determine if the NP366-specific CD8+ T cells induced by immunization with live virus incorporated in gelatin gel were functional and produced IFNγ when stimulated with peptide antigen. Total splenocytes from mice that were either un-manipulated or immunized with virus alone, virus incorporated in gelatin, or gelatin alone, were stimulated in culture for six hours in the presence of NP366-374 peptide and brefeldin A. Production of IFNγ was assessed by flow-cytometry using fluorochrome-coupled anti-IFNγ antibodies that bind to the IFNγ accumulated inside the cells over the six hours of stimulation with peptide. Upon in vitro stimulation with NP366-374 peptide, about 8% of CD8+ T cells in the spleen of mice immunized with live virus incorporated in gelatin gel, and about 17% of CD8+ T cells in the spleen of mice immunized with live virus alone, produced IFNγ (FIG. 6C). In contrast, in mice immunized with gelatin gel only as well as in un-manipulated mice, less than 1% of CD8+ splenocytes produced IFNγ. In the absence of peptide stimulation, the percentage of CD8+ T cells producing IFNγ was below 1% for all samples. Therefore, the gelatin polymer facilitated release of the virus in the body and did not alter the immunogenicity of the virus since mice treated with this mode of immunization mounted robust immune responses of magnitudes similar to mice immunized with live virus alone.

To test the safety of live virus delivered in gelatin polymer, Rag−/−γc−/− mice were SQ immunized with 100 μl gelatin containing 10 HAU live virus or gelatin alone. All Rag−/−γc−/− mice remained healthy and did not lose weight over a 30-day time period, therefore confirming that gelatin gels are not toxic and the SQ route of delivery does not cause active viral infection.

Collagen Gel

A preliminary study on the delivery of gel encapsulated virus particles was conducted by subcutaneously delivering to mice a mass of gelatin polymer containing the virus and the carrier material. Collagen was explored as a carrier material because of its well established use as a biopolymer in other settings. The rate of degradation of collagen, for the release of trapped virus particles, can be controlled by controlling the degree of crosslinking of the collagen and by the choice of the crosslinking agent (van Wachem et al., 1991, Biomaterials 12:215-223). However, the cytotoxicity of the crosslinker (van Luyn et al., 1992, J. Biomed. Materials Res. 26:1091-111-; van Luyn et al., 1992, Biomaterials 13:1017-1024) and exacerbating effects on calcification (Golomb et al., 1987, Am. J. Pathol. 127:122-130) renders some crosslinking agents less useful. Furthermore, since crosslinking involves covalent bond formation between the crosslinking agent and the amino acid moieties of the collagen fibrils, the surface protein molecules of the virus particles might be expected to participate in the same process which would result in inactivation of the virus. Despite these potential issues, viral particles suspended in a matrix of a collagen gel have been shown to improve the efficiency of DNA transfection and protein expression and delivery (Schek et al., 2004, Molecular Therapy 9:130-138; Gu et al., 2004, Molecular Therapy 9:699-711).

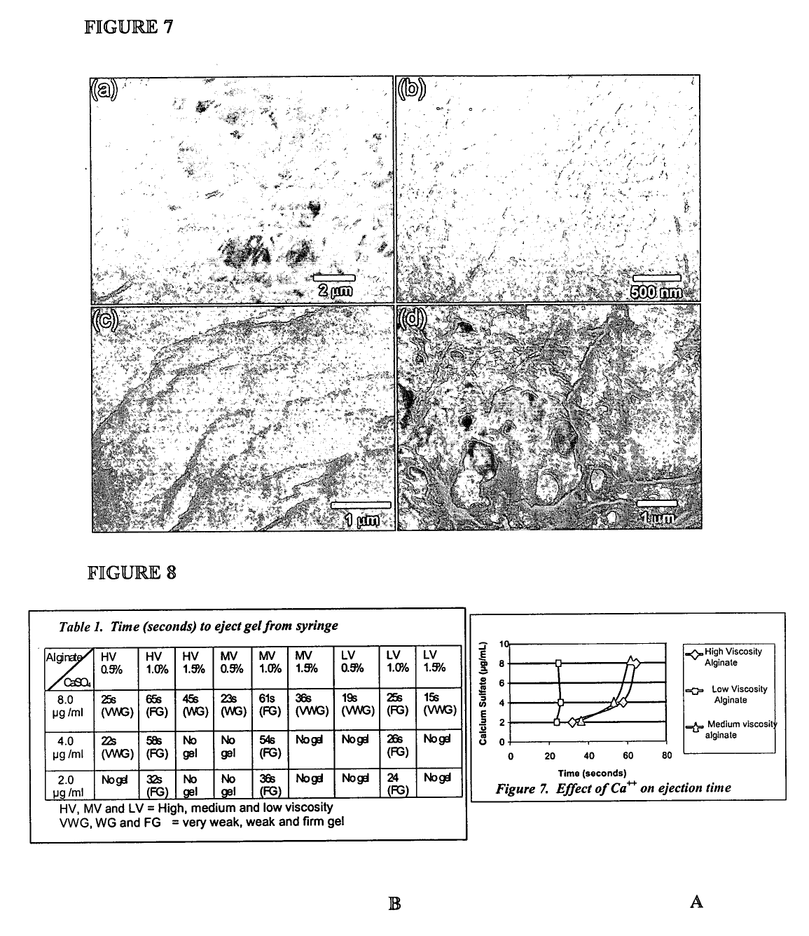

In order to deliver live virus into the body of a mammal using the SQ or ID routes, collagen gels were synthesized inside stainless needles (30G½). Stock collagen is an acidic solution that remains liquid at about 4° C. after mixing with 10×PBS buffer and 1N sodium hydroxide to achieve neutral pH. This solution was transferred to the needles using a syringe and the loaded needles were incubated at 37° C. for about 30 minutes whereupon the collagen became a fibrous gel. Two approaches were investigated in preparing the gels and loading them into the syringe. One involved preparation of the gels in microcentrifuge tubes (bulk casting) and in the other, the neutralized collagen solution was taken up in the needle prior to incubation and the gels were therefore cast inside the needle (microcasting). The tips of the needles were capped using a thick slab of polydimethysiloxane (PDMS) which prevented leakage of material from the needle during the incubation period. Electron micrographs of hydrogels prepared by microcasting of 6 mg/ml and 10 mg/ml collagen were taken using a Philips XL30 Environmental Scanning Electron Microscope (ESEM) and the results are shown in FIGS. 7a and 7b, respectively. For scanning electron microscopy (SEM) sample preparation, the gels were freeze dried after extrusion through the needle and coated with a thin film of platinum. Pore sizes were found to be in the order of 500 nm to 21 μm when the collagen concentration was maintained at 6 mg/ml. When 10 mg/ml collagen was used as the starting material, there was a significant increase in packing density of the fibrils as compared with the packing density when 6 mg/ml collagen was used as a starting material (FIG. 7a). Pore sizes for gels formed with the 10 mg/ml collagen are about less than or equal to 200 nm. These results demonstrate that the release rate of virus particles can be controlled depending on the properties of the starting collagen material.

It has been shown that environmental scanning electron microscopy (ESEM) can be used to study hydrodynamic processes at the nanoscale (Babu et al., 2005, Miccrofluiducs and Nanofluidics, 1:284-288; Rossi et al., 2004, Nano Letters 4:989-993). The ability to image biomaterials in the presence of water facilitates the examination of structures at nanoscale while maintaining the sample in its natural state and reducing destructive sample preparation time. In order to elucidate the difference in the morphology of gels examined by Scanning Electron Microscopy in the traditional mode and in wet mode (ESEM), gels were prepared by microcasting using a 10 mg/ml collagen starting solution. Micrographs were taken in both systems. A comparison of the micrographs revealed that the gel morphology as assessed by the two methods was the same (FIGS. 7b and 7c), despite the differences in sample preparation and examining mode. As an example of the flexibility inherent in polymer blends, in FIG. 7d, there is shown freeze dried blends of collagen (10 mg/ml) and polyethylene glycol (MW 20,000 from Alfa Aesar) at a ratio 1:4 (collagen:PEG). Pore sizes of the hydrogel vary between 100 and 500 nm which represents an increase when compared with the single component collagen gel.

These preliminary results demonstrate that it is possible to modulate pore size in collagen gels by manipulating the original density and also by using polymer blends/copolymers (i.e., the PEG blends described herein). Pore size is a critical factor that given the ability of a polymer to release virus and facilitate subsequent dendritic cell uptake, an event that is crucial to the elicitation of a protective immune response.

Alginate Polymers

In order to develop a hydrogel encapsulation system that allows a virus-containing gel to set in situ inside the needle of an inoculation syringe, the use of alginate polymers was explored. Direct injection of the gel by either the SQ or ID route, should facilitate administration of live virus to the mammal with minimal risk of aerosolization and therefore minimal exposure to the personnel administering the dose.

Three sodium alginate powder samples were obtained from FMC Biopolymer. Alginate solutions of varying viscosities can be synthesized by varying the concentration and type of alginate. The strength of a cross-linked alginate gel can be varied by also varying the concentration of the cross-linking ion. A 0.5%, 1% or 1.5% (w/v) solution of sodium alginate was prepared in deionized water (DI) and the solution was sterilized by filtration through a 0.45 μm syringe filter. Gelation was initiated by the addition of a solution containing sodium metaphosphate, which was sterilized by autoclaving. Alginate (5 ml) was transferred to a 50 ml conical tube and 8, 4 or 2 μg/ml of CaSO₄ (from a slurry of 0.4 g/ml) was added. The contents of the tube were shaken vigorously to ensure complete mixing of the alginate and the CaSO₄ slurry, and a small aliquot of the mixture was drawn up into a 5 ml syringe fitted with a 22G needle, enough to just fill the needle, and leaving a 1 ml air space between the needle end and the plunger. The syringe was clamped in a vertically-mounted syringe pump and the contents of the syringe were expelled by applying a constant rate of 63 ml/min. This procedure ensured consistent pressure on all formulations. The time required to eject the gelled alginate and a visual inspection of the gel was recorded. The results are shown in FIG. 8B.

Alginate viscosity decreases with a decrease in molecular weight. Thus, the gel became easier to eject (e.g. at 8 μg/ml CaSO₄ and 1% gel, the time for ejection was HV=65 s>MV=62 s>LV=25 s). As the ratio of alginate to calcium decreased, a limit was often reached at which no gel was formed (e.g. passing down the HV/0.5% column: no gel is formed when the calcium concentration drops to 2.0 μg/ml). The limiting calcium concentration at which no gel formed, was lowest for the high viscosity alginate. The exception was at 1% alginate which formed gels at calcium concentrations for all three alginates. In all cases at 1% alginate concentration, a firm gel was formed that was ejected from the syringe with increasing ease as the calcium ion concentration decreased. When the results for the 1% solutions are plotted on a graph (FIG. 8A), it is evident that the high and medium viscosity alginates behave in a very similar manner, and the low viscosity alginate was removed from the needle with greater ease.

Release of Virus from Alginate Polymers

To assess the potential for virus release from alginate polymers, the following experiments were performed. The data described herein provide parameters for varying the mechanical properties of alginate gels. Based on these data, it was determined that quantum dots (QDots) would be a convenient model for encapsulation of virus. QDots are 10-20 nm diameter nanocrystals composed of an inner core of CdSe/ZnS and an outer shell with functional groups for ease of conjugation. Not only are the size and shape of QDots ideal for the applications described herein, but also the QDots fluoresce intensely, making them easy to detect and quantitate.

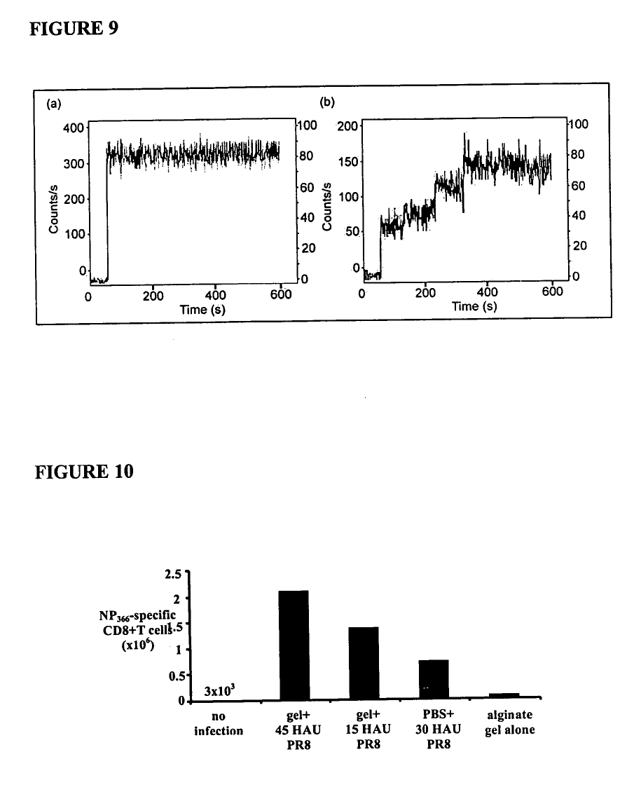

Using a mixture of 4 μg/ml CaSO₄ to 1% alginate gel containing QDots, samples were drawn into a 22G needle of a 5 ml syringe, and were allowed to set as described elsewhere herein. The gel pellet containing QDots was then injected into 4 ml of phosphate buffered saline (PBS) held in a cuvette housed in a PTI fluorimeter. Fluorescence was monitored from the time of injection. In FIGS. 9a and 9b, there is shown two distinct profiles of fluorescence that were evident. Low viscosity alginate released its contents as soon as it was injected into the PBS. The fluorescence intensity rose to a maximum at 50 seconds, the time of injection. High viscosity gels released their contents more gradually after injection at 50 seconds, completing release after 400 seconds.

In Vitro Assay for Active Virus Release from Gel-Encapsulated Virus

An in vitro assay was developed to test for release of virus particles from alginate gels. MDCK cells are an adherent cell line that supports the growth of various viruses including influenza viruses. In preliminary studies, MDCK cells were cultured with live virus entrapped in alginate polymer for five days and were then tested in a hemagglutination assay using a chicken red blood cell suspension to assess infection. In negative controls, using alginate alone, no virus was detected (absence of hemagglutination). In contrast, intense hemagglutination was observed for 50% and 75% virus in alginate gels, as well as in the positive controls where 6 HAU and 3 HAU of virus in solution was assessed. These studies demonstrate that the infectivity of influenza virus is preserved within the alginate gels and that live, infectious is released from the gels at 37° C.

Virus Entrapped in Alginate Gels

Alginates gels have been approved by the U.S. Food and Drug Administration (FDA) as being generally regarded as safe (GRAS). The data presented herein establish that live influenza virus entrapped in an alginate gel is immunogenic and elicits both cytotoxic CD8+ T cells and virus neutralizing antibodies in a mammal (FIG. 10). In FIG. 10, it can be seen that live PR8 virus administered in alginate gels potently stimulated the CD8+ T cell response in mice. Large numbers of pulmonary NP₃₆₆-specific CD8+ T cells were elicited in animals that were inoculated subcutaneously with live virus in alginate gels and then were subsequently rechallenged with X31 influenza virus. Lungs from groups of mice that were either unmanipulated (that is, control mice), or from mice inoculated subcutaneously with PR8 virus alone, alginate alone or PR8 live virus encapsulated in alginate, were analyzed seven days following intranasal rechallenge with X31 influenza virus. Single cell suspensions obtained from the mice were stained with anti-CD8 antibodies and MHC class I/NP₃₆₆₋₃₇₄ tetrameric complexes that recognize the immunodominant NP₃₆₆₋₃₇₄ peptide. The stained cells were analyzed by flow cytometry. The values shown in FIG. 10 represent the average obtained from two mice per group.

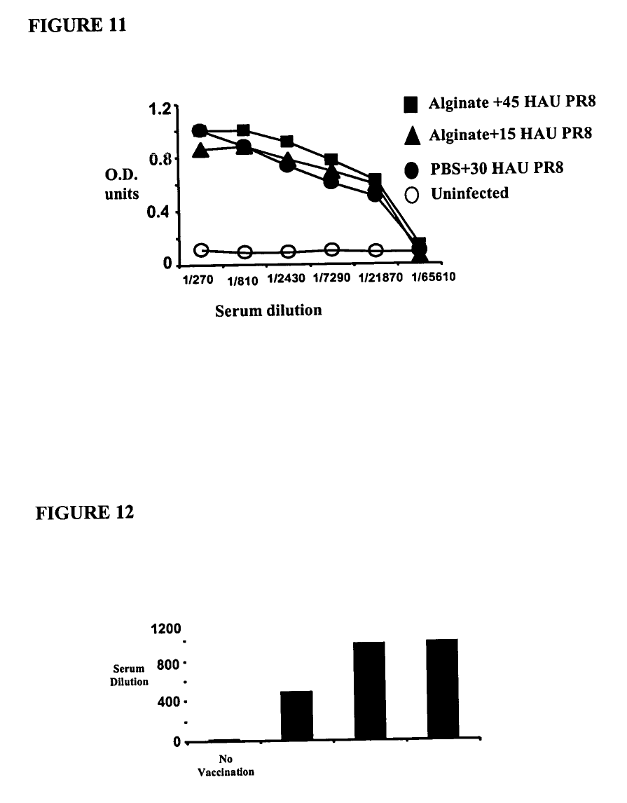

In addition, IgG antibody responses in the mice were also elicited at high titers as shown in FIG. 11. Anti-PR8 antibodies present in the serum of C57BL/6 mice immunized with PR8 virus alone or encapsulated in alginate gel, were detected by ELISA using PR8 virus as capturing antigen. The 1/270 initial serum dilution was further diluted in 3-fold serial dilutions and added to plate-bound PR8 virus. Uninfected animals exhibited no antibody responses to PR8 virus.

Most preventive vaccines work by generating neutralizing antibodies in the host that block subsequent infection with the virus. The data shown in FIG. 12 establish that live influenza virus trapped in an alginate gel elicits high titers of neutralizing antibodies mice in addition to the cytotoxic CD8+ T cell response shown in FIG. 10. In FIG. 12, a hemagglutination inhibition assay was performed using PR8, chicken red blood cells and dilutions of sera obtained from immunized animals. The maximum serum dilution that exhibited hemagglutination inhibition is shown for animals immunized with live virus in alginate gel. Sera from non-immunized animals exhibited no hemagglutination inhibition.

Nanotubes

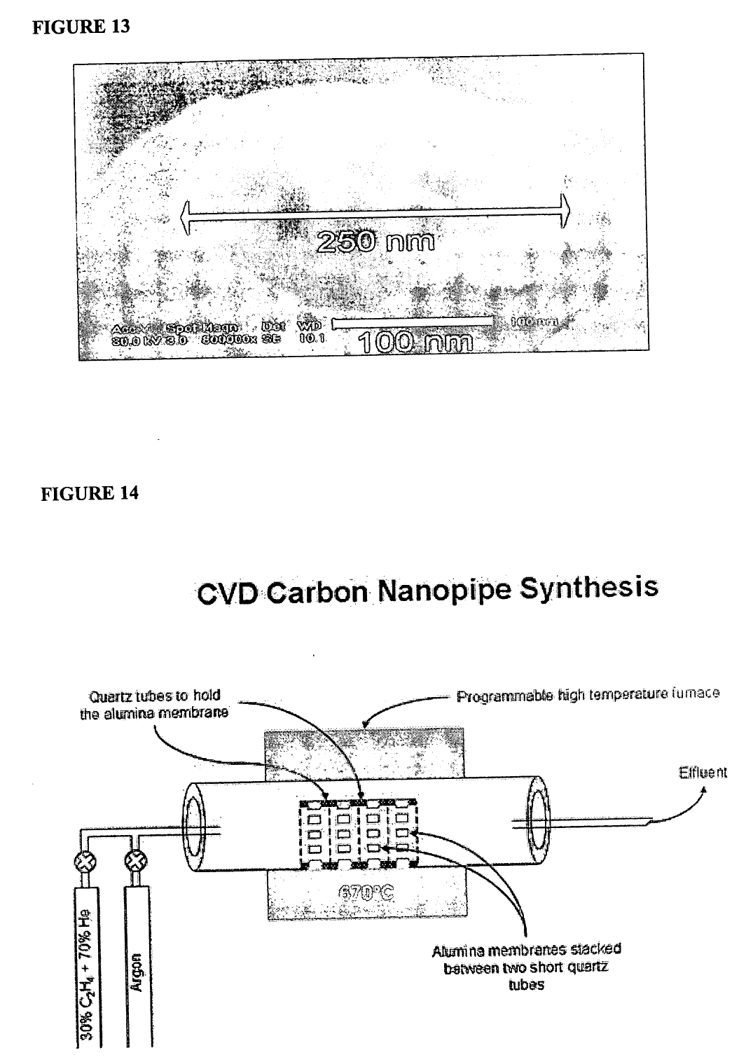

As discussed elsewhere herein, a preferable vaccine is one that is loaded into nanotubes. It is possible to synthesize carbon nanotubes of various diameters (50-250 nm) (Bradley et al., 2003, Chemistry Preprint Server, Miscell.: 1-6, CPS: chemistry/0303002; Babu et al., Microfluidics and Nanofluidics 1:284-288; Rossi et al., 2004, Nano Letters 4:989-993). Templates for the synthesis of nanotubes having larger diameters (250 nm) are commercially available. Larger diameter nanotubes facilitate the generation of structures that can retain and release particles the size of an influenza virus. The type of nanotube that is preferred in the present application is known as a multi-wall nanotube (MWNT), although this type of tube lacks the proper crystalline structure normally found in nanotubes synthesized using a metal catalyzed Chemical Vapor Deposition (CVD) process. Nanotubes were synthesized by following the template assisted method established by Miller et al. (Miller et al., 2001, J. Amer. Chem. Soc. 123:12335-12342). In FIG. 13 there is shown the cross section of a typical large diameter nanotube synthesized using the methods described herein.

The ability to load carbon nanotubes with magnetic (Korneva et al., 2005, Nano Letters, 5:879-884) or fluorescent nanoparticles Kim et al., 2005, Nano Letters 5:873-878) has been recently demonstrated in a rather simple methodology, despite the small tube diameter of 275+/−25 nm and the resulting capillary action. Nanotubes for these experiments were synthesized by CVD process. The evaporation rate of the solvent within the tube has been shown to be much higher than the displacement of the trapped particle resulting in precipitation of the particles along the walls of the nanotubes (Kim et al., 2005, Nano Letters, 5:873-878). This is a safety measure designed by nature. Thus virus that is loaded inside these nanotubes will not freely diffuse out of the lumen of the tube. These results of these studies establish that a 250 nm diameter nanotube can be loaded with various aqueous solutions by condensation (Babu et al., supra; Rossi et al., supra). Preferably, in the present invention, nanotubes will be loaded with fluids that are more viscous than water. However this is not expected to generate any problems because fluids that are as viscous as glycerol and ethylene glycol have been successfully loaded in other settings (Kim et al., supra).

The experiments presented herein should be considered to be applicable to the use of nanoencapsulated live virus in polymers such as collagen matrices, alginates, gelatin polymers that release virus at body temperature. These approaches render airborne viruses safer for use as vaccines while taking advantage of the very low doses needed to induce protective immune responses in vaccinated individuals.

Synthesis of Aligned Carbon Nanotubes

In brief, an alumina membrane (Whatman Anodisc 13 mm diameter, and a 250 nm pore size) placed in a quartz reaction vessel acts as the template for the carbon nanotubes to grow. A tube furnace capable of reaching at least 1000° C. will be used to crack a mixture of ethylene and argon gas flowing at a rate of 20 sccm over the alumina membrane. The decomposition of ethylene gas at 670° C. results in deposition of carbon around the inner walls of the alumina membrane; the thickness of the deposited carbon layer thus depends on the process time. For the intended purpose a reaction time of 6 hours will be adequate. The layer of carbon on the sides of the membrane will be removed using mild sonnication (47 kHz, bath sonnicator). The membranes with carbon nanotubes must be completely soaked in 1M NaOH for at least twelve hours for the complete removal of template. The nanotubes can be removed from the suspension after template removal by filtering though polycarbonate membrane filters with 1 micron pores (SPI Supplies). A schematic representation of the process is shown in FIG. 14.

Nanotube Loading

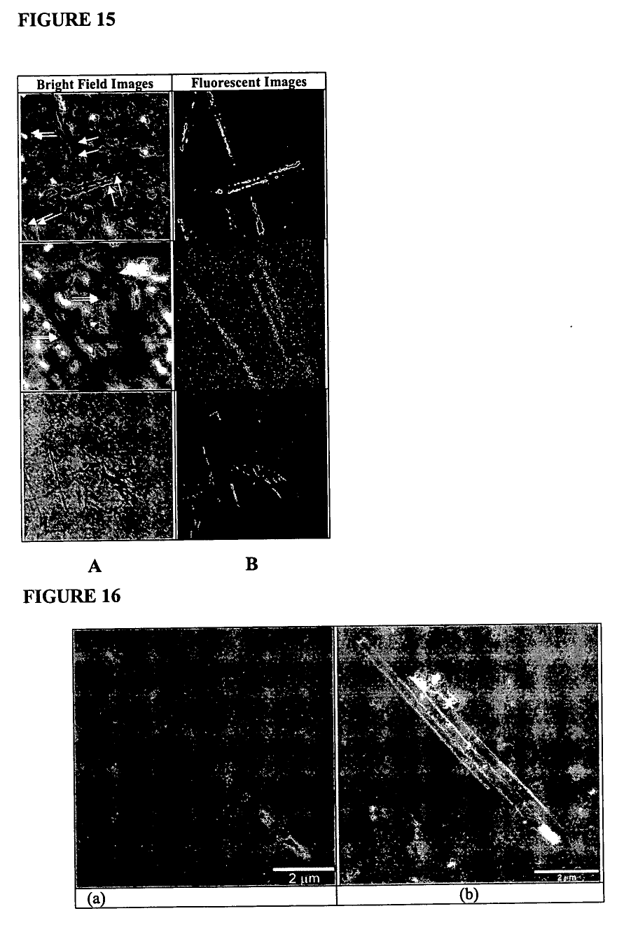

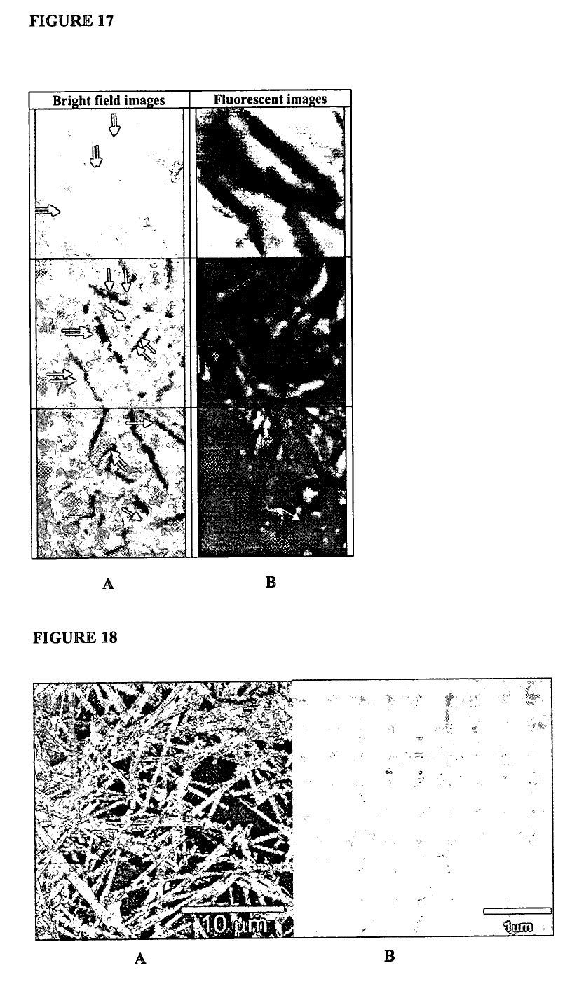

A loading process has been developed that allows for the efficient loading of liquids and gels into nanotubes. The data presented herein establish that the loading of virus containing alginate gels into nanotubes is feasible. Alginate gels that contain QDots that are approximately the size of viral particles (˜50-100 nm diameter) were loaded into nanotubes. As can be seen in FIGS. 15 and 16, QDots were loaded inside the nanotubes. Note that the nanotubes were transparent and QDot fluorescence was transmitted through the tube wall. For controls, nanotubes that were mixed with a solution of alginate and QDots but that did not undergo the loading process are shown (FIG. 17). In this latter setting, the QDots fluoresce as background outside the tubes but not inside. The details of these experiments are as follows.

FIG. 15 depicts confocal images of carbon nanotubes filled with sodium alginate and quantum dots are shown. Nanotubes were mixed with alginate gel that contained 50 nm QDots and then underwent a loading procedure. The presence of fluorescence inside the tubes indicates the loading of the tube with QDot containing gel. Arrows point to individual tubes.

FIG. 16 is a series of scanning electron microscope images of carbon nanotubes containing sodium alginate and quantum dots (QDots) are shown. Gel and QDots can be clearly seen inside the tubes (FIGS. 16a and 16b). The scale bars on both images measures 2 μm.

FIG. 17 depicts that nanotubes simply mixed with alginate gels that contain QDots do not load. Confocal images of carbon nanotubes in the presence of sodium alginate and QDots are shown. Nanotubes were mixed with alginate gel that contained 50 nm QDots but were not subjected to the loading procedure. Fluorescence from QDots is seen as background and not inside the nanotubes which appear black. Arrows point to individual tubes.

Sonnication Breaks Nanotubes to Lengths Smaller than 500 nm

An important component of the bioactive agent release strategy is the ability to break the nanotubes using ultrasound so that controlled release of the bioactive agent occurs in the animal in which it is administered. In order to release the bioactive agent, nanotubes should be broken into sizes where the capillary forces within the tubes facilitate the release of both the gel and live virus into the surrounding tissues. In FIG. 18, data are shown that demonstrate that 10-12 μm long nanotubes that are sonnicated at 1.36 MHz for 30 seconds break into much smaller tubes having a size of about less than 1 μm. The skilled artisan will know, based on the experiments presented herein, how to optimize the experimental conditions to modulate the release of the contents of the nanotube into the surrounding tissues. In FIG. 18, there is shown scanning electron micrographs of carbon nanotubes (FIG. 18a) before sonnication (×1000 magnification) and (FIG. 18b) after sonnication (×10,000 magnification) at 1.36 MHz for 30 seconds. Before sonnication, nanotubes were 10-20 μm long (FIG. 18a). After sonnication, the nanotubes were less than 1 μm long (FIG. 18b).

Alginate Hydrogel

Methods for in situ gelation described herein can be optimized and expanded to investigate the effects on gel properties with use of multi valent cations other than Ca²⁺, for example insoluble salts such as barium carbonate, phosphate or sulfate, and aluminum phosphate or hydroxide, will replace calcium sulfate. Alginates of different molecular weights and composition (guluronic:manuronic acid ratio in polymer backbone). Effects on ease of injection, and release profiles of QDots (easily quantifiable model for viral particles) will preface studies of the efficacy of virus delivery using both in vitro hemagglutination assays and eventually in vitro studies in mouse (vide supra). Parallel studies can be conducted using HA or other mixtures of a;ginate and chitosan, or other combinations known to those skilled in the art.

Collagen Gel

Based on the preliminary data on collagen gel synthesis, hydrogels can be synthesized and characterized hydrogels for their ability to encapsulate, store and release virus at a specific rate when administered in vivo. Gels with increasing densities of collagen starting material (4, 6, 8 and 10 mg/ml) can be synthesized according to the procedures disclosed elsewhere herein, and can be cast by both bulk casting and micro casting. The gels are dried following the standard protocols for critical point drying (Philips XL30) and examined under a scanning electron microscope (SEM) for variations in pore dimensions. Collagen gels are also be synthesized in the presence of calculated amount of fluorescent quantum dot particles (QDots). These QDots can be purchased from Quantum Dot Corporation or Evident Technologies. The gel is separated and washed several times with PBS buffer before using. The rate of diffusion of QDots from the gel is estimated by following the emission spectra of the QDots, while maintaining the gel at 37° C. A UV-Visible spectrometer with temperature controllable cuvette holder is used to collect the emission spectra at various times. The rate of diffusion data and the porosity information obtained from the SEM is compared and used to determine the optimum collagen concentration that meets the required release rate of trapped virus particles. After arriving at the optimum collagen concentration, polyethylene glycol (PEG, M.W. 10000, 8000, 6000 and 1,000) is added as an additive during the synthesis process. Collagen/PEG composites are synthesized in the presence of QDots using the same procedures described herein. The concentration of PEG can be varied by changing the ratio of collagen to PEG in the following order, 1:0.5, 1:1, 1:2, and 1:4. The ratio that retains the original rate of diffusion but increases homogeneity and minimizes phase separation can be chosen as the formulation for in vivo testing. Morphology changes in the hydrogel after addition of PEG can be monitored by SEM.

Gelatin hydrogel

Based on the data presented herein on gelatin for use to encapsulate virus, hydrogels can be synthesized and characterized that can encapsulate, store and release the virus when administered in vivo. Gelatin, lyophilized and γ-irradiated is used to prepare the hydrogels as disclosed elsewhere herein. The concentration of the gelatin in water is varied (from 1 to 3% w/v) in order to attain the optimum release rate. Loss of water from the hydrogel during storage results in shrinking of the gelatin hydrogel. This can be reduced by adding calculated quantities of PEG (1 to 10% w/w of gelatin) oligomers (M.W. 400 to 1000) during gel formation. Gel strength and measurement of physical dimensions is used to determine the rate of shrinking. Periodic measurement of the viscosity and cross-linking density of the gels using oscillatory rheometric testers (Bohlin Controlled Stress Rheometer) should reveal changes in the viscoelastic property of the gels. By placing the gel between two discs as the bottom one oscillates in this instrument, the frequency is transmitted through the gel and the torque is measured by the upper disc. The identical procedure as that described herein for collagen and QD's can be used with gelatin and QD's to determine the release rate of viral size particles.

Methods for Assessing Safety

To exclude the potential for aerosolization, aerosol creation needs to be measured. For this purpose 50 nm QDot loaded polymer gels can be squirted onto a Petri dish. Air samples are collected using an SKS Biosampler which is designed to work with a sonic flow pump and is especially efficient at collecting bioaerosols. The Biosampler is made of glass and is equipped with three tangential nozzles which act as critical orifices, each permitting 4.2 liters of ambient air to pass through resulting in a total flow rate of 12.5 liters per minute. Bioaerosols are captured in a swirling liquid trap of PBS, water or culture media. The Biosampler uses a high-volume sonic flow pump to trap airborne viable microorganisms for subsequent analysis. Sampling for aerosols is conducted at 10 cm and 100 cm directly in front of the surface on which polymer gel is squirted and represents the maximum potential release. Air samples are passed through 20 ml PBS (for QDots) or culture medium (for virus) to collect particles. Solutions are concentrated 10-fold and assayed. For QDot experiments fluorescence is measure with a fluorimeter. For studies with virus, polymers are considered safe when collected samples exhibit no hemagglutination in a chicken RBC assay following culture of 2 ml (10-fold concentrated) samples for five days in MDCK cells. The immunogenicity and safety of virus loaded polymer gels is assessed as described herein for non-encapsulated virus. The same or procedures easily identified by those skilled in the art can be employed for any bioactive agent. The use of quantum dots is a simple example of an optical biosensor of the size of a virus particle. One may use any tag, optical, magnetic, electrical of the size of the viral particle to assess aerosol formation.

Nanotubes

To investigate nanotubes as potential delivery vehicles for gel-trapped bioactive agent the following experiments can be performed. The nanofluidic loading and release characteristics of polymer gel loaded nanotubes and the conditions for controlled release of polymer from nanotubes can be assessed as described below. Safety and immunogenicity is assessed as described elsewhere herein. Nanotubes loaded with polymer gel containing bioactive agent is an alternative strategy for agent delivery from the conventional syringe and needle method of delivery that is commonly in use.

Fluids and polymers are loaded into nanotubes by soaking the nanotubes on a polycarbonate 200 nm membrane in the appropriate liquids for 1 minute and then applying a mild vacuum. This process is repeated five times. When silver nanoparticles of 50 nm size were used, this process resulted in a loading efficiency of 3040% of colloidal particles.

To investigate the nanofluidic loading of the nanotubes, 50 nm QDots encapsulated at various concentrations in gels are used to quantitate loading efficiency of gels. The fluorescence intensity of nanotubes after extensive washing is used to measure QDot concentrations. The number of nanotubes that have loaded is quantitated by confocal microscopy. The nanotube walls are transparent with respect to UV light, therefore fluorescence within the tube can be visualized. To load the polymer gels, gels are mixed with nanotubes before polymerization and a number of cycles with vacuum are applied. Nanotubes are then exposed to crosslinking agents or to temperature to catalyze cross-linking depending on the polymer used.

The release characteristics of gel loaded nanotubes is investigated with or without sonnication (20 kHz-1.3 MHz). Gels containing 50 nm QDots are assessed using a fluorimeter to measure released QDots. However, confocal microscopy facilitates the quantitation of number of nanotubes that are loaded with QDots, or that have released them following sonnication.

Following determination of optimal conditions of loading QDot containing polymer into nanotubes, live virus containing polymer loaded nanotubes is examined in vitro and in vivo as described elsewhere herein and not repeated here.

Delivery systems based on nanotubes have several advantages when compared with prior art methods. Because the virus particles are trapped inside the tube, accidental spillage cannot result and therefore the safety of personnel in the vicinity is enhanced. The evaporation rate of the solvent within the tube has been shown to be much higher than the displacement of the trapped particle resulting in precipitation of the particles along the walls of the nanotubes, creating a safety measure designed by nature.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A vaccine comprising a CD8+ T cell immunoprotective and/or antibody immunoprotective amount of virus, wherein said virus induces an immunoprotective CD8+ T cell and/or antibody response in an animal following administration of said virus to said animal by a route that does not cause disease in said animal.

2. A vaccine comprising a CD8+ T cell immunoprotective amount of virus, wherein said virus induces an immunoprotective CD8+ T cell response in an animal following administration of said virus to said animal by a route that does not cause disease in said animal.

3. The vaccine of claim 1, wherein said virus is a live virus.

4. The vaccine of claim 1, wherein said virus is an attenuated virus.

5. The vaccine of claim 1, wherein said virus is a killed virus.

6. The vaccine of claim 1, wherein said virus is a respiratory virus.

7. The vaccine of claim 1, wherein said virus is selected from the group consisting of an orthomyxovirus, a paramyxovirus, a coronavirus, a picornavirus, respiratory syncytial virus, measles virus, adenovirus, a parvovirus, and adenovirus, a calicivirus, an astrovirus, Norwalk virus, an arenavirus, a flavivirus, a filovirus, a hantavirus, an alphavirus, a retrovirus and a lentivirus.

8. The vaccine of claim 7, wherein said virus is an orthomyxovirus.

9. The vaccine of claim 8, wherein said orthomyxovirus is an influenza virus.

10. The vaccine of claim 9, wherein said influenza virus is influenza virus type A.

11. The vaccine of claim 10, wherein said influenza virus type A has a hemagglutinin antigen (HA) selected from the group consisting of H1, H2, H3, H4, H5, H6, H7, H8, H9, H10, H11, H12, H13, H14, H15 and H16.

12. The vaccine of claim 10, wherein said influenza virus type A has a neuraminidase antigen (NA) selected from the group consisting of N1, N2, N3, N4, N5, N6, N7, N8 and N9.

13. The vaccine of claim 10 wherein said influenza virus type A has a HA:NA antigenic profile selected from the group consisting of H5N1, H9N2, H7N1, H7N2, H7N3, H7N7, H2N2, H1N1, H1N2 and H3N2.

14. The vaccine of claim 10 comprising a low dose of said influenza virus type A.

15. The vaccine of claim 14, wherein said low dose of influenza type A virus is from 0.001 to 5000 hamagglutination units (HAU) of virus.

16. The vaccine of claim 15, wherein said low dose of influenza virus type A is from 0.005 to 500 HAU of virus.

17. The vaccine of claim 16, wherein said low dose of influenza virus type A is from 0.01 to 100 HAU of virus.

18. The vaccine of claim 1, wherein said animal is a mammal.

19. The vaccine of claim 18, wherein said mammal is a human.

20. The vaccine of claim 1, wherein said virus comprises a combination of two or more of member selected from the group consisting of a live virus, an attenuated virus, and a killed virus.

21. The vaccine of claim 1, wherein said route is a non-natural route.

22. The vaccine of claim 21, wherein said route is selected from the group consisting of subcutaneous, intradermal, intramuscular, mucosal and oral.

23. A kit comprising the vaccine of claim 1.

24. A vaccine comprising a CD8+ T cell immunoprotective and/or antibody immunoprotective amount of virus, wherein said virus induces an immunoprotective CD8+ T cell and/or antibody response in an animal following administration of said virus to said animal by a route that does not cause disease in said animal, and further wherein said virus is associated with an encapsulation vehicle.

25. A vaccine comprising a CD8+ T cell immunoprotective amount of virus, wherein said virus induces an immunoprotective CD8+ T cell response in an animal following administration of said virus to said animal by a route that does not cause disease in said animal, and further wherein said virus is associated with an encapsulation vehicle.

26. The vaccine of claim 24, wherein said virus is encapsulated in said encapsulation vehicle.

27. The vaccine of claim 24 wherein said virus is associated with a nanotube, a lipsome or a protein prior to being encapsulated in said encapsulation vehicle.

28. The vaccine of claim 24, wherein said encapsulation vehicle comprises one or more members selected from the group consisting of a gel, a liquid or a powder.

29. The vaccine of claim 28, wherein said encapsulation vehicle is loaded into a nanotube.

30. The vaccine of claim 24, wherein said encapsulation vehicle comprises a polymer.

31. The vaccine of claim 30, wherein said polymer is not toxic when administered to an animal.

32. The vaccine of claim 30, wherein said polymer is associated with said virus thereby delaying release of said virus into the surrounding environment.

33. The vaccine of claim 30, wherein said polymer is a gel.

34. The vaccine of claim 33, wherein said gel comprises collagen.

35. The vaccine of claim 33, wherein said gel is a hydrogel.

36. The vaccine of claim 35, wherein said hydrogel is selected from the group consisting of an alginate, gelatin, chitosan and hyaluronic acid.

37. The vaccine of claim 35, wherein said hydrogel is selected from the group consisting of polyvinylpyrrolidone and carboxymethyl cellulose.

38. The vaccine of claim 33, wherein said gel comprises a combination of one or more of collagen, alginate, gelatin, chitosan, hyaluronic acid, polyvinylpyrrolidone and carboxymethyl cellulose.

39. The vaccine of claim 33, wherein said gel is crosslinked.

40. The vaccine of claim 24, further comprising an additive.

41. The vaccine of claim 40, wherein said additive is polyethylene glycol.

42. The vaccine of claim 24, wherein said encapsulation vehicle comprises a microcapsule.

43. The vaccine of claim 24, wherein said encapsulation vehicle comprises a nanocapsule.

44. The vaccine of claim 24, wherein said encapsulation vehicle comprises a nanotube.

45. The vaccine of claim 44, wherein said nanotube has a diameter of 500 nm or less.

46. The vaccine of claim 24, wherein said encapsulation vehicle comprises a combination of one or more of a solution, a powder or a gel.

47. The vaccine of claim 24, wherein said virus is a live virus.

48. The vaccine of claim 24, wherein said virus is an attenuated virus.

49. The vaccine of claim 24, wherein said virus is a killed virus.

50. The vaccine of claim 24, wherein said virus is a respiratory virus.

51. The vaccine of claim 24, wherein said virus is selected from the group consisting of an orthomyxovirus, a paramyxovirus, a coronavirus, a picomavirus, respiratory syncytial virus, measles virus, adenovirus, a parvovirus, and adenovirus, a calicivirus, an astrovirus, Norwalk virus, an arenavirus, a flavivirus, a filovirus, a hantavirus, an alphavirus, a retrovirus and a lentivirus.

52. The vaccine of claim 51, wherein said virus is an orthomyxovirus.

53. The vaccine of claim 52, wherein said orthomyxovirus is an influenza virus.

54. The vaccine of claim 53, wherein said influenza virus is influenza virus type A.

55. The vaccine of claim 54, wherein said influenza virus type A has a hemagglutinin antigen (HA) selected from the group consisting of H1, H2, H3, H4, H5, H6, H7, H8, H9, H10, H11, H12, H13, H14, H15 and H16.

56. The vaccine of claim 54, wherein said influenza virus type A has a neuraminidase antigen (NA) selected from the group consisting of N1, N2, N3, N4, N5, N6, N7, N8 and N9.

57. The vaccine of claim 54, wherein said influenza virus type A has a HA:NA antigenic profile selected from the group consisting of H5N1, H9N2, H7N1, H7N2, H7N3, H7N7, H2N2, H1N1, H1N2 and H3N2.

58. The vaccine of claim 54 comprising a low dose of said influenza virus type A.

59. The vaccine of claim 58, wherein said low dose of influenza type A virus is from 0.001 to 5000 hamagglutination units (HAU) of virus.

60. The vaccine of claim 59, wherein said low dose of influenza virus type Amis from 0.005 to 500 HAU of virus.

61. The vaccine of claim 60, wherein said low dose of influenza virus type A is from 0.01 to 100 HAU of virus.

62. The vaccine of claim 24, wherein said animal is a mammal.

63. The vaccine of claim 62, wherein said mammal is a human.

64. The vaccine of claim 24, wherein said virus comprises a combination of two or more of a live virus, an attenuated virus, and a killed virus.

65. The vaccine of claim 24, wherein said route is a non-natural route.

66. The vaccine of claim 65, wherein said route at least one member selected from the group consisting of subcutaneous, intradermal, intramuscular, mucosal and oral.

67. A kit comprising the vaccine of claim 24.

68. A device for delivery of a vaccine to an animal, said device comprising (a) a CD8+ T cell immunoprotective and/or antibody amount of virus, wherein said virus induces an immunoprotective CD8+ T cell and/or antibody response in an animal following administration of said virus to said animal by a non-natural route, (b) a delivery device for delivering said vaccine to said animal.

69. A device for delivery of a vaccine to an animal, said device comprising (a) a CD8+ T cell immunoprotective amount of virus, wherein said virus induces an immunoprotective CD8+ T cell response in an animal following administration of said virus to said animal by a non-natural route, (b) a delivery device for delivering said vaccine to said animal.

70. The device of claim 68, wherein said delivery device comprises a hollow tube.

71. The device of claim 70, wherein said hollow tube has a tapered end.

72. The device of claim 71, wherein said delivery device comprises a needle.

73. The device of claim 70, wherein said hollow tube is optionally attached to a plunging device.

74. The device of claim 73, wherein said plunging device is a syringe.

75. The device of claim 68, wherein said delivery device is a gene gun.

76. The device of claim 68, wherein said delivery device is a catheter.

77. The device of claim 68, wherein said delivery device is a patch.

78. The device of claim 68, wherein said delivery device is an inhaler.

79. The device of claim 68, wherein said delivery device is a mucosal applicator.

80. The device of claim 68, further comprising an encapsulation vehicle.

81. The device of claim 80, wherein said virus is encapsulated in said encapsulation vehicle.

82. The device of claim 80 wherein said virus is associated with a nanotube, a lipsome or a protein prior to being encapsulated in said encapsulation vehicle.

83. The device of claim 80, wherein said encapsulation vehicle is one or more members selected from the group consisting of a gel, a liquid or a powder.

84. The device of claim 83, wherein said encapsulation vehicle is loaded into a nanotube.

85. The device of claim 80, wherein said encapsulation vehicle comprises a polymer.

86. The device of claim 85, wherein said polymer is not toxic when administered to an animal.

87. The device of claim 86, wherein said polymer is associated with said virus thereby delaying release of said virus into the surrounding environment.

88. The device of claim 85, wherein said polymer is a gel.

89. The device of claim 88, wherein said gel comprises collagen.

90. The device of claim 88, wherein said gel is a hydrogel.

91. The device of claim 90, wherein said hydrogel is selected from the group consisting of an alginate, gelatin, chitosan and hyaluronic acid.

92. The device of claim 90, wherein said hydrogel is selected from the group consisting of polyvinylpyrrolidone and carboxymethyl cellulose.

93. The device of claim 88, wherein said gel comprises a combination of one or more of collagen, alginate, gelatin, chitosan, hyaluronic acid, polyvinylpyrrolidone and carboxymethyl cellulose.

94. The device of claim 88, wherein said gel is crosslinked.

95. The device of claim 80, further comprising an additive.

96. The device of claim 95, wherein said additive is polyethylene glycol.

97. The device of claim 80, wherein said encapsulation vehicle comprises a microcapsule.

98. The device of claim 80, wherein said encapsulation vehicle comprises a nanocapsule.

99. The device of claim 80, wherein said encapsulation vehicle comprises a nanotube.

100. The device of claim 99, wherein said nanotube has a diameter of 500 nm or less.

101. The device of claim 80, wherein said encapsulation vehicle comprises a combination of one or more of a solution, a powder or a gel.

102. The device of claim 80, wherein said virus is a live virus.

103. The device of claim 80, wherein said virus is an attenuated virus.

104. The device of claim 80, wherein said virus is a killed virus.

105. The device of claim 80, wherein said virus is a respiratory virus.

106. The device of claim 80, wherein said virus is selected from the group consisting of an orthomyxovirus, a paramyxovirus, a coronavirus, a picornavirus, respiratory syncytial virus, measles virus, adenovirus, a parvovirus, and adenovirus, a calicivirus, an astrovirus, Norwalk virus, an arenavirus, a flavivirus, a filovirus, a hantavirus, an alphavirus, a retrovirus and a lentivirus.

107. The device of claim 106, wherein said virus is an orthomyxovirus.

108. The device of claim 107, wherein said orthomyxovirus is an influenza virus.

109. The device of claim 108, wherein said influenza virus is influenza virus type A.

110. The device of claim 109, wherein said influenza virus type A has a hemagglutinin antigen (HA) selected from the group consisting of H1, H2, H3, H4, H5, H6, H7, H8, H9, H10, H11, H12, H13, H14, H15 and H16.

111. The device of claim 109, wherein said influenza virus type A has a neuraminidase antigen (NA) selected from the group consisting of N1, N2, N3, N4, N5, N6, N7, N8 and N9.

112. The device of claim 109, wherein said influenza virus type A has a HA:NA antigenic profile selected from the group consisting of H5N1, H9N2, H7N1, H7N2, H7N3, H7N7, H2N2, H1N1, H1N2 and H3N2.

113. The device of claim 109 comprising a low dose of said influenza virus type A.

114. The device of claim 113, wherein said low dose of influenza type A virus is from 0.001 to 5000 hamagglutination units (HAU) of virus.

115. The device of claim 114, wherein said low dose of influenza virus type Amis from 0.005 to 500 HAU of virus.

116. The device of claim 115, wherein said low dose of influenza virus type A is from 0.01 to 100 HAU of virus.

117. The device of claim 80, wherein said animal is a mammal.

118. The device of claim 117, wherein said mammal is a human.

119. The device of claim 80, wherein said virus comprises at least two members selected from the group consisting of a live virus, an attenuated virus, and a killed virus.

120. The device of claim 80, wherein said route is a non-natural route.

121. The device of claim 120, wherein said route is at least one members selected from the group consisting of subcutaneous, intradermal, intramuscular, mucosal and oral.

122. A method of making a vaccine comprising a CD8+ T cell immunoprotective and/or antibody immunoprotective amount of virus, said method comprising combining a CD8+ T cell and/or antibody immunoprotective amount of a virus with an encapsulation vehicle, thereby making said vaccine.

123. A method of making a vaccine comprising a CD8+ T cell immunoprotective amount of virus, said method comprising combining an immunoprotective amount of a virus with an encapsulation vehicle, thereby making said vaccine.

124. A method of eliciting a CD8+ T cell immunoprotective and/or antibody immune response in an animal, said method comprising administering to said animal a vaccine comprising a CD8+ T cell and/or antibody immunoprotective amount of virus, whereby a CD8+ T cell and/or antibody immune response is elicited in said mammal.

125. A method of eliciting a CD8+ T cell immune response in an animal, said method comprising administering to said animal a vaccine comprising a CD8+ T cell immunoprotective amount of virus, whereby a CD8+ T cell immune response is elicited in said animal.

126. The method of claim 124, wherein said animal is a mammal.

127. The method of claim 126, wherein said mammal is a human.

128. A method of protecting an animal against infection by a virus, said method comprising administering to said animal a vaccine comprising a CD8+ T cell immunoprotective and/or antibody immunoprotective amount of said virus, whereby a CD8+ T cell and/or antibody immune response is elicited in said animal thereby protecting said animal against said infection.

129. A method of protecting an animal against infection by a virus, said method comprising administering to said animal a vaccine comprising a CD8+ T cell immunoprotective amount of said virus, whereby a CD8+ T cell immune response is elicited in said animal thereby protecting said animal against said infection.

130. The nethod of claim 128, wherein said animal is a mammal.

131. The method of claim 130, wherein said mammal is a human.

132. A method of preventing a virus infection in an animal, said method comprising administering to said animal a vaccine comprising a CD8+ T cell immunoprotective and/or antibody immunoprotective amount of said virus, whereby a CD8+ T cell and/or antibody immune response is elicited in said animal thereby preventing a virus infection in said animal.

133. A method of preventing a virus infection in an animal, said method comprising administering to said animal a vaccine comprising a CD8+ T cell immunoprotective amount of said virus, whereby a CD8+ T cell immune response is elicited in said animal thereby preventing a virus infection in said animal.

134. The method of claim 132, wherein said animal is a mammal.

135. The method of claim 134, wherein said mammal is a human.

136. A method of treating a virus infection in an animal, said method comprising administering to said animal a vaccine comprising a CD8+ T cell immunoprotective and/or antibody immunoprotective amount of said virus, whereby a CD8+ T cell and/or antibody immune response is elicited in said animal thereby treating said animal.

137. A method of treating a virus infection in an animal, said method comprising administering to said animal a vaccine comprising a CD8+ T cell immunoprotective and/or antibody immunoprotective amount of said virus, whereby a CD8+ T cell and/or antibody immune response is elicited in said animal thereby treating said animal.

138. The method of claim 136, wherein said animal is a mammal.

139. The method of claim 138, wherein said mammal is a human.

140. A composition comprising a CD8+ T cell immunoprotective and/or antibody immunoprotective amount of a bioactive agent, wherein said bioactive agent induces an immunoprotective CD8+ T cell and/or antibody response in an animal following administration of said bioactive agent to said animal by a route that does not cause disease in said animal.

141. A composition comprising a CD8+ T cell immunoprotective amount of bioactive agent, wherein said bioactive agent induces an immunoprotective CD8+ T cell response in an animal following administration of said bioactive agent to said animal by a route that does not cause disease in said animal.

142. The composition of claim 140, wherein said route is a non-natural route.

143. The composition of claim 140, wherein said route is selected from the group consisting of subcutaneous, intradermal, intramuscular, mucosal and oral.

144. The composition of claim 140, wherein said bioactive agent is encapsulated in said encapsulation vehicle.

145. The composition of claim 140, wherein said bioactive agent is associated with a nanotube, a lipsome or a protein prior to being encapsulated in said encapsulation vehicle.

146. The composition of claim 140, wherein said encapsulation vehicle comprises at least one member selected from the group consisting of a gel, a liquid or a powder.

147. The composition of claim 146, wherein said encapsulation vehicle is loaded into a nanotube.

148. The composition of claim 147, wherein said encapsulation vehicle comprises a polymer.

149. The composition of claim 148, wherein said polymer is not toxic when administered to an animal.

150. The composition of claim 148, wherein said polymer is associated with said bioactive agent thereby delaying release of said bioactive agent into the surrounding environment.

151. The composition of claim 148, wherein said polymer is a gel.

152. The composition of claim 140, wherein said bioactive agent is selected from the group consisting of a microorganism, and a protein.

153. A method of enhancing safety when administering a bioactive agent to an animal, said method comprising administering a composition comprising a CD8+ T cell immunoprotective and/or antibody immunoprotective amount of a bioactive agent to said animal, wherein said bioactive agent induces an immunoprotective CD8+ T cell and/or antibody response in said animal following administration of said bioactive agent by a route that does not cause disease in said animal and further wherein said bioactive agent is encapsulated in an encapsulation vehicle.

154. A method of enhancing safety when administering a bioactive agent to an animal, said method comprising administering to said animal a composition comprising a CD8+ T cell immunoprotective amount of bioactive agent, wherein said bioactive agent induces an immunoprotective CD8+ T cell response in said animal following administration of said bioactive agent by a route that does not cause disease in said animal and further wherein said bioactive agent is encapsulated in an encapsulation vehicle.

155. The method of claim 153, wherein said bioactive agent is selected from the group consisting of a microorganism and a protein.

156. A composition comprising a biologically effective amount of a bioactive agent, wherein said bioactive agent induces a desired response in an animal while reducing risk in an animal following administration of said bioactive agent to said animal by a route that does not cause disease in said animal.

157. The composition of claim 156, wherein said route is a non-natural route.

158. The composition of claim 157, wherein said route is selected from the group consisting of subcutaneous, intradermal, intramuscular, mucosal and oral.

159. The composition of claim 156, wherein said bioactive agent is encapsulated in an encapsulation vehicle.

160. The composition of claim 156, wherein said bioactive agent is associated with a nanotube, a liposome or a protein prior to being encapsulated in said encapsulation vehicle.

161. The composition of claim 156, wherein said encapsulation vehicle is at least one member selected from the group consisting of a gel, a liquid or a powder.

162. The composition of claim 159, wherein said encapsulation vehicle is loaded into a microcapsule, nanocapsule or nanotube.

163. The composition of claim 159, wherein said encapsulation vehicle comprises a polymer.

164. The composition of claim 163, wherein said polymer is not toxic when administered to an animal.

165. The composition of claim 163, wherein said polymer is associated with said bioactive agent thereby delaying release of said bioactive agent into the surrounding environment.

166. The composition of claim 163, wherein said polymer is a gel.

167. The composition of claim 156, wherein said bioactive agent is selected from the group consisting of a microorganism, and a protein.

168. A method of enhancing safety when administering a bioactive agent to an animal, said method comprising administering to an animal a composition comprising an amount of a bioactive agent that induces a desired response while reducing risk in an animal, wherein the route of administration of said bioactive agent is a route that does not cause disease in said animal, and further wherein said bioactive agent is encapsulated in an encapsulation vehicle thereby enhancing safety when administering said bioactive agent.
